(19) United States
(12) Patent Application Publication
(10) Pub. No.: US 2024/0353801 A1
(43) Pub. Date: Oct. 24, 2024

(54) WEARABLE ELECTRONIC DEVICE COMPRISING BIOMETRIC SENSOR

(71) Applicant: Samsung Electronics Co., Ltd., Suwon-si (KR)

(72) Inventors: Jungsoo KIM, Suwon-si (KR); Donghoo JANG, Suwon-si (KR); Jongkuck SON, Suwon-si (KR); Junhui LEE, Suwon-si (KR); Jonggwan JUNG, Suwon-si (KR); Sungnam KANG, Suwon-si (KR); Soonwan CHUNG, Suwon-si (KR)

(21) Appl. No.: 18/750,574

(22) Filed: Jun. 21, 2024

Related U.S. Application Data
(63) Continuation of application No. PCT/KR2022/021398, filed on Dec. 27, 2022.

(30) Foreign Application Priority Data
Jan. 28, 2022 (KR) 10-2022-0013533
Apr. 6, 2022 (KR) 10-2022-0043068
Nov. 25, 2022 (KR) 10-2022-0160691

(51) Int. Cl. G04G 17/02; G04G 21/02

(52) U.S. Cl. CPC G04G 17/02; G04G 21/025

(57) ABSTRACT

A wearable electronic device is provided. The wearable electronic device includes a metal frame, a plurality of electrodes positioned on the metal frame so as to be in contact with a body of a user and configured to acquire biometric information by forming an electrical signal path together with the body of the user being in contact, a metal strap connected to the metal frame, and an insulating structure, wherein the insulating structure may be configured to electrically separate the metal strap from the metal frame to reduce leaking of the electrical signal to the metal frame through the metal strap.

### CROSS-REFERENCE TO RELATED APPLICATION(S)

This application is a continuation application, claiming priority under § 365 (c), of an International application No. PCT/KR2022/021398, filed on Dec. 27, 2022, which is based on and claims the benefit of a Korean patent application number 10-2022-0013533, filed on Jan. 28, 2022, in the Korean Intellectual Property Office, of a Korean patent application number 10-2022-0043068, filed on Apr. 6, 2022, in the Korean Intellectual Property Office, and of a Korean patent application number 10-2022-0160691, filed on Nov. 25, 2022, in the Korean Intellectual Property Office, the disclosure of each of which is incorporated by reference herein in its entirety.

### BACKGROUND

### 1. Field

The disclosure relates to a wearable electronic device including a biometric sensor.

### 2. Description of Related Art

Nowadays, in addition to hand held type electronic devices such as smartphones, wearable type electronic devices worn by users on their bodies, such as smart watches, smart glasses, or earbuds, are being developed.

Furthermore, as people's interest in health increases, the demand for wearable electronic devices with health care functions is increasing.

One of the healthcare functions is to provide biometric information about a user's body composition.

The above information is presented as background information only to assist with an understanding of the disclosure. No determination has been made, and no assertion is made, as to whether any of the above might be applicable as prior art with regard to the disclosure.

### SUMMARY

As a method for providing information about a user's body composition, there is bioelectric impedance analysis (BIA). The bioelectric impedance analysis is a method of measuring a user's body composition based on the current applied to a user's body. Since a wearable device is worn on the user's body, the wearable device may include a part (e.g., a strap) that is in contact with the user's body in addition to a part (e.g., electrode) for applying electric current to measure biometric information. The part may form an unintended and abnormal current path, which may reduce the accuracy of biometric information measurement.

Aspects of the disclosure are to address at least the above-mentioned problems and/or disadvantages and to provide at least the advantages described below. Accordingly, an aspect of the disclosure is to provide a wearable electronic device with improved accuracy of biometric information measurement.

Additional aspects will be set forth in part in the description which follows and, in part, will be apparent from the description, or may be learned by practice of the presented embodiments.

In accordance with an aspect of the disclosure, a wearable electronic device is provided. The wearable electronic device includes a metal frame, a plurality of electrodes positioned on the metal frame to be in contact with a user's body and configured to form an electrical signal path with the user's body in contact to acquire biometric information, a metal strap connected to the metal frame, and an insulating structure, and the insulating structure is configured to electrically separate the metal strap and the metal frame to reduce leaking of the electrical signal to the metal frame through the metal strap.

Another aspect of the disclosure is to improve accuracy of biometric information measurement of a wearable electronic device.

Another aspect of the disclosure is to improve accuracy of biometric information measurement through an insulating structure that electrically separates a housing and a coupling member of the wearable electronic device.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a front perspective view of an electronic device according to an embodiment of the disclosure;

FIG. 2 is a rear perspective view of the electronic device according to an embodiment of the disclosure;

FIG. 3 is an exploded perspective view of the electronic device according to an embodiment of the disclosure;

FIG. 4A shows a state in which the electronic device is worn on a user's body according to an embodiment of the disclosure;

FIG. 4B is a conceptual diagram showing a method of acquiring biometric information through a first biometric sensor according to an embodiment of the disclosure;

FIG. 5A is a view showing a fixing member according to an embodiment of the disclosure;

FIG. 5B is a view showing a coupling member to which the fixing member is coupled, according to an embodiment of the disclosure;

FIG. 6 is a view showing a coupling member to which a fixing member is coupled, according to an embodiment of the disclosure;

FIG. 7 is a view showing a coupling member to which a fixing member is coupled, according to an embodiment of the disclosure;

FIG. 8A is a view showing a part of an electronic device according to an embodiment of the disclosure;

FIG. 8B shows examples of insulating structures of the electronic device according to an embodiment of the disclosure;

FIG. 9 shows a housing of an electronic device according to an embodiment of the disclosure;

FIG. 10 shows a side member and a printed circuit board of an electronic device according to an embodiment of the disclosure;

FIG. 11A shows an electronic device according to an embodiment of the disclosure;

FIG. 11B shows electrodes of the electronic device according to an embodiment of the disclosure;

FIG. 12 is a block diagram of an electronic device according to an embodiment of the disclosure;

FIG. 13 shows an electronic device in a network environment according to an embodiment of the disclosure;

FIG. 14A is a view showing a coupling member according to an embodiment of the disclosure;

FIG. 14B is a view showing the coupling member according to an embodiment of the disclosure;

FIG. 14C is a view showing the coupling member according to an embodiment of the disclosure;

FIG. 14D is a view showing the coupling member according to an embodiment of the disclosure;

FIG. 15A is a view showing an end link to which an adhesive member is applied, according to an embodiment of the disclosure;

FIG. 15B is a view showing a coupling member in which a strap is coupled to the end link, according to an embodiment of the disclosure;

FIG. 16A is a view showing a coupling member including a blocking member according to an embodiment of the disclosure;

FIG. 16B is a view showing an example of the blocking member according to an embodiment of the disclosure;

FIG. 17A is a view showing an example of a first region according to an embodiment of the disclosure;

FIG. 17B is a view showing an example of the first region according to an embodiment of the disclosure;

FIG. 18A is a view showing an end link of a coupling member according to an embodiment of the disclosure;

FIG. 18B is a cross-sectional view taken along line A-A′ in FIG. 18A according to an embodiment of the disclosure;

FIG. 18C is a view showing the coupling member in which a strap is coupled to the end link according to an embodiment of the disclosure; and

FIG. 19 is a view showing a method of inspecting insulation of a coupling member according to an embodiment of the disclosure.

Throughout the drawings, it should be noted that like reference numbers are used to depict the same or similar elements, features, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a Wi-Fi chip, a Bluetooth® chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

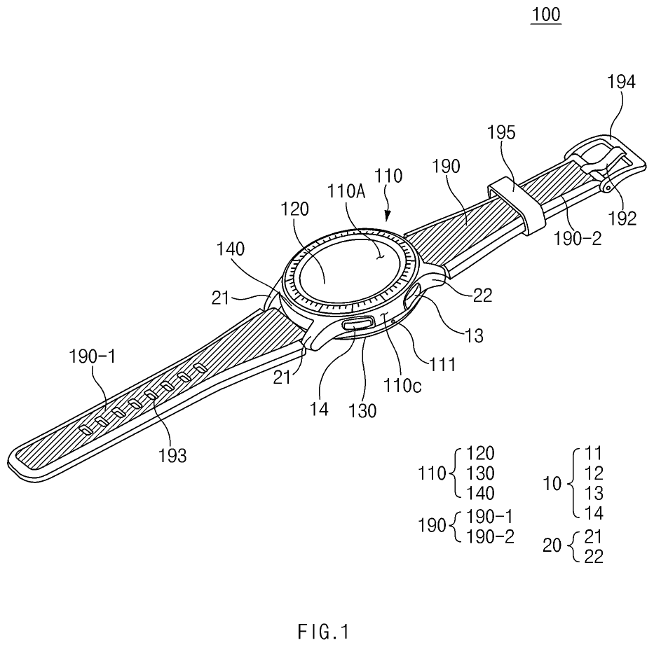

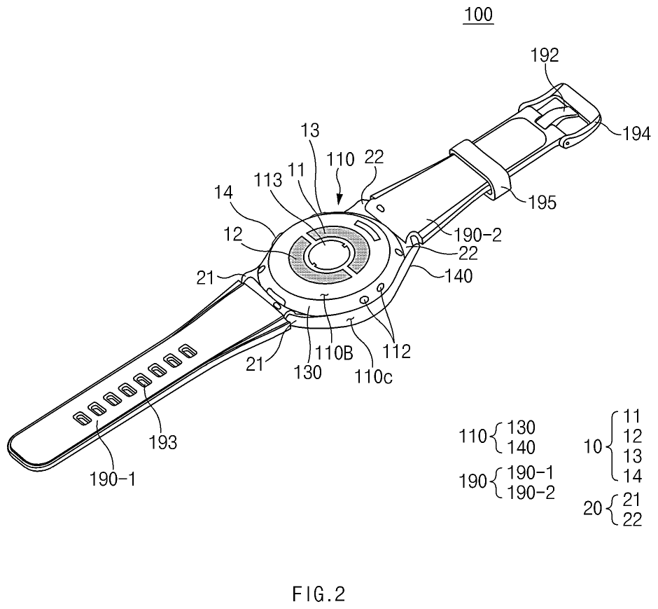

FIG. 1 is a front perspective view of an electronic device according to an embodiment of the disclosure. FIG. 2 is a rear perspective view of the electronic device according to an embodiment of the disclosure.

Referring to FIGS. 1 and 2, an electronic device 100 according to an embodiment may be a wearable electronic device. For example, the electronic device 100 may be a watch-type electronic device (e.g., a smart watch) capable of being worn on a part of a user's body (e.g., a wrist or an ankle). However, the electronic device according to various embodiments disclosed herein is not limited to the shown embodiment.

In one embodiment, the electronic device 100 may include a housing 110. The housing 110 may form at least a portion of the exterior of the electronic device 100. The housing 110 may include a front member 120 (e.g., a front plate or front cover), a side member 140 (e.g., a frame), and/or a rear member 130 (e.g., a back plate or back cover). The front member 120, the side member 140, and/or the rear member 130 may be coupled to each other.

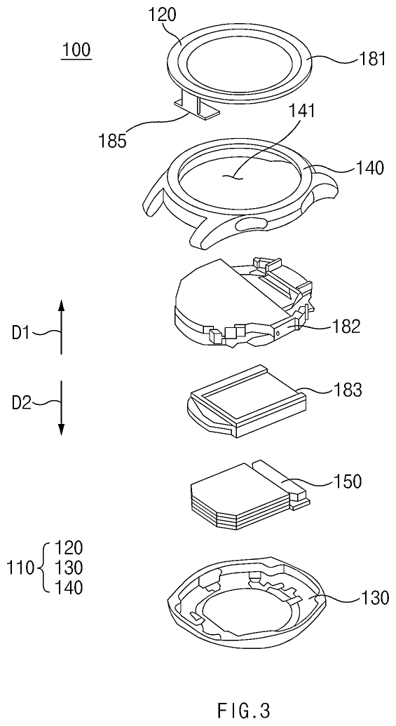

In one embodiment, the housing 110 may include a structure in which the side member 140 surrounds a space between the front member 120 and the rear member 130. The housing 110 may provide an inner space allowing other components (e.g., a display 181, a bracket 182, a circuit board 150, and/or a battery 183 in FIG. 3) of the electronic device 100 to be accommodated through a coupled structure of the front member 120, the side member 140, and/or the rear member 130.

In one embodiment, the housing 110 may include a front surface 110A, a rear surface 110B facing an opposite direction of the front surface 110A, and a side surface 110C surrounding a space between the front surface 110A and the rear surface 110B. The front member 120 may at least partially define the front surface 110A. The side member 140 may at least partially define the side surface 110C. The rear member 130 may at least partially define the rear surface 110B. In the disclosure, the housing 110 may be referred to as a structure defining at least a portion of the front surface 110A, the rear surface 110B, and the side surface 110C.

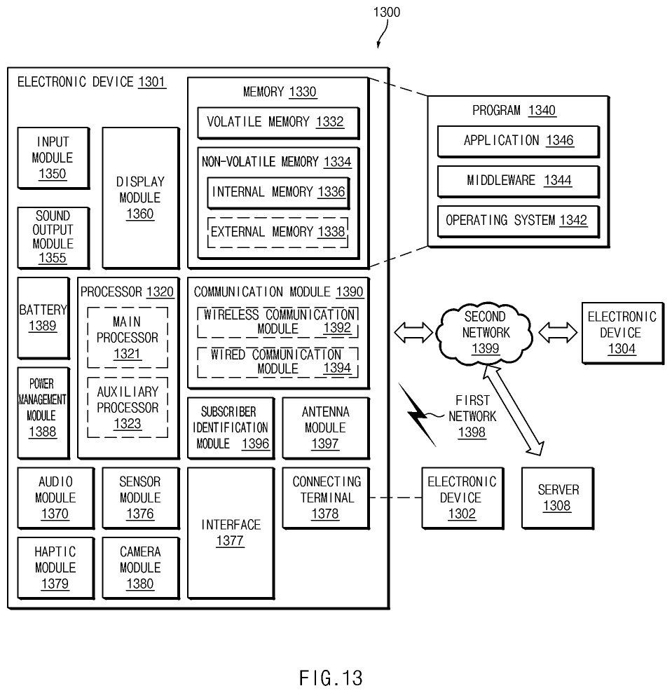

In one embodiment, the electronic device 100 may include a microphone hole 111 formed in the rear member 130 and a microphone (or acoustic sensor) (e.g., an audio module 1370 in FIG. 13) disposed adjacent to the microphone hole 111. The microphone may be disposed inside the housing 110. The microphone may be configured to detect sound propagating from the outside through the microphone hole 111. Optionally, a plurality of microphones and a plurality of microphone holes 111 may be provided to detect sound from various directions.

In one embodiment, the electronic device 100 may include a speaker hole 112 formed in the rear member 130 and a speaker disposed adjacent to the speaker hole 112 (e.g., a sound output module 1355 in FIG. 13). The speaker may be disposed inside the housing 110. The speaker may be configured to output sound. The sound output from the speaker may be propagated to the outside through the speaker hole 112. Alternatively, the speaker of the electronic device 100 may be configured such that the speaker hole 112 is omitted (e.g., a piezo speaker).

The microphone hole 111 and/or the speaker hole 112 are not limited to the shown embodiment. For example, the speaker hole 112 and the microphone hole 111 may be implemented as one hole. For another example, the microphone hole 111 and/or the speaker hole 112 may be disposed in a component (e.g., the side member 140) other than the rear member 130.

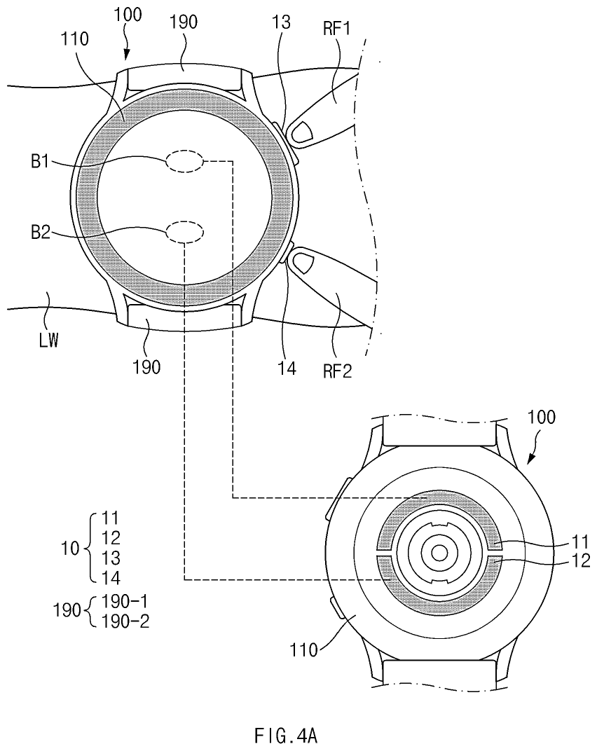

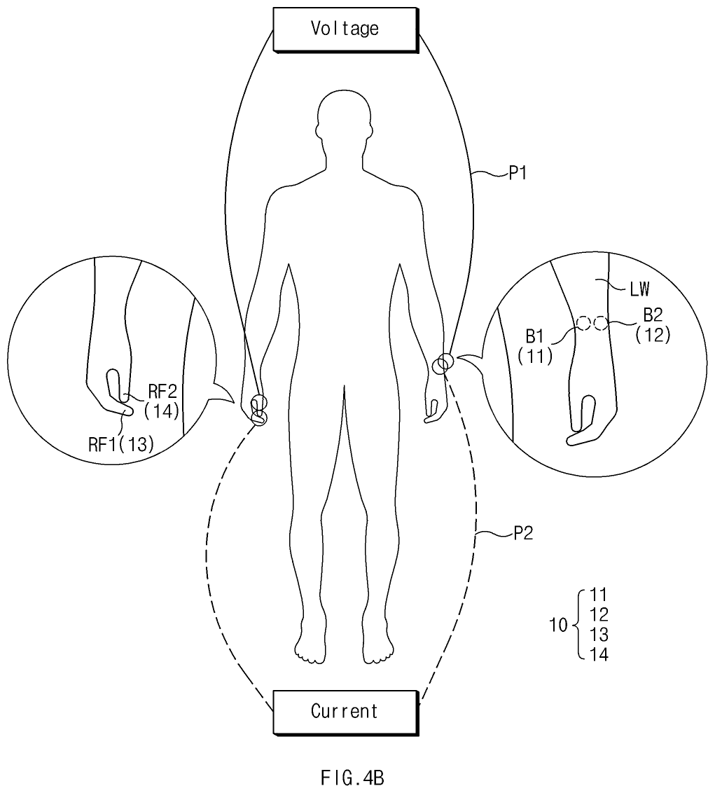

In one embodiment, the electronic device 100 may include a first biometric sensor 10. The first biometric sensor 10 may generate an electrical signal or data value corresponding to an external environmental state. For example, the first biometric sensor 10 may include a bioelectric impedance sensor for acquiring biometric information about components of a user's body (e.g., a body fat content). The first biometric sensor 10 may include a first electrode 11, a second electrode 12, a third electrode 13, and a fourth electrode 14. The first electrode 11 and the second electrode 12 may be disposed on the rear member 130 to define a portion of the rear surface 110B. The first electrode 11 and the second electrode 12 may be spaced apart from each other on the rear member 130, and may come into contact with the user's body when the user wears the electronic device 100. The third electrode 13 and the fourth electrode 14 may be disposed on the side member 140 to be exposed through the side surface 110C. The first electrode 11, the second electrode 12, the third electrode 13, and the fourth electrode 14 may be made of an electrically conductive material. The first biometric sensor 10 may measure the composition of the user's body based on the electrical resistance detected through the first electrode 11, second electrode 12, third electrode 13, and fourth electrode 14 in contact with the user's body. A method of acquiring biometric information using the first biometric sensor 10 will be described in detail below with reference to FIGS. 4A and 4B.

In one embodiment, the third electrode 13 and the fourth electrode 14 may be configured as a key input device (e.g., side keys) for receiving user input. For example, the third electrode 13 and the fourth electrode 14 may be configured as button members capable of being rotated and/or pressed by a user's manipulation. However, the key input device is not limited thereto, and the key input devices implemented through the third electrode 13 and/or the fourth electrode 14 may be of another type, for example, be implemented in the form of a soft key on the touch-sensitive display device (e.g., the display 181 in FIG. 3) of the electronic device 100.

In one embodiment, the electronic device 100 may include a second biometric sensor module 113. The second biometric sensor module 113 may be disposed inside the housing 110, and at least a portion of the second biometric sensor module 113 may be visually exposed to the rear surface 110B of the housing 110 through a partial region of the rear member 130. The second biometric sensor module 113 may generate an electrical signal or data value corresponding to the internal operational state or external environmental state of the electronic device 100. For example, the second biometric sensor module 113 may include a sensor (e.g., a heart rate sensor and a blood oxygen saturation sensor) for acquiring biometric information on the user. For example, the second biometric sensor module 113 may include at least one light emitting unit and at least one light receiving unit. The at least one light emitting unit may be made up of, for example, a light emitting diode (LED) or a laser diode (LD). The at least one light receiving unit may be made up of, for example, a photodiode or an image sensor. The second biometric sensor module 113 may detect a biometric signal by reflecting light emitted by the light emitting unit onto the user's body (e.g., the wrist) and detecting light incident on the light receiving unit. For example, the second biometric sensor module 113 may detect the reflectance (or transmittance) of light according to the change in blood volume within the blood vessel due to contraction/relaxation of the heart (or change in blood vessel volume due to change in blood volume) and the electronic device 100 may acquire biometric information about the heart rate based on the detected biometric signal. For another example, the second biometric sensor module 113 may detect the reflectance (or transmittance) of light according to the amount of oxygen in the blood, and the electronic device 100 may acquire biometric information about oxygen saturation in the blood based on the detected biometric signal. However, the configuration for the second biometric sensor module 113 to acquire biometric information is not limited to the above-described example, and various methods that are easy for those skilled in the art may be applied.

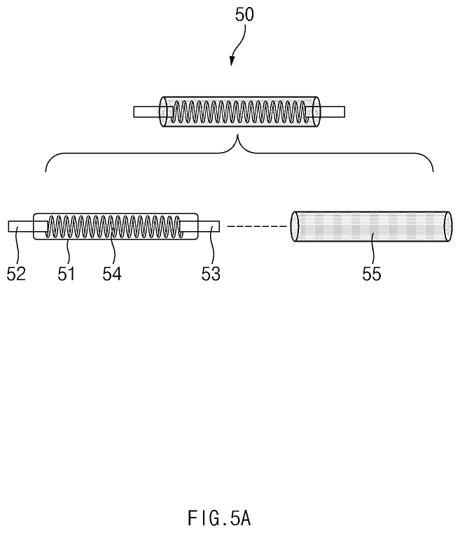

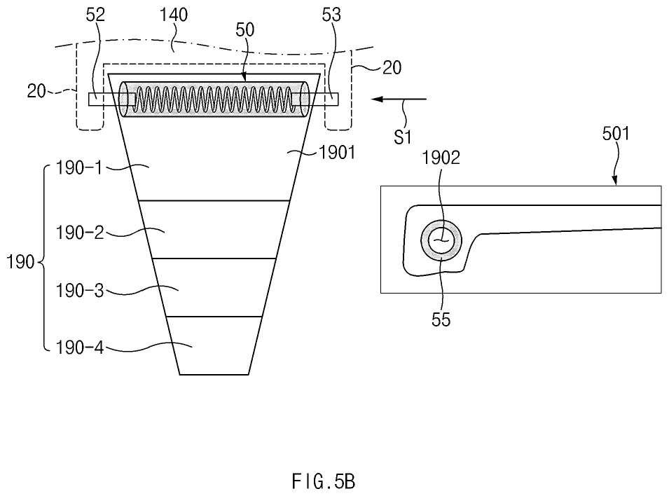

In one embodiment, the electronic device 100 may include a coupling member 190 configured to surround a part of the user's body (e.g., wrist). The coupling member 190 may be coupled to the housing 110. The coupling member 190 may be coupled to lugs 20 formed on the side member 140. For example, the coupling member 190 may be coupled to the lugs 20 through a fixing member (e.g., a fixing member 50 in FIG. 5A). The lugs 20 may protrude from the side member 140 to define a space in which ends of the coupling member 190 are to be accommodated. When the coupling member 190 is accommodated in the lugs 20, the fixing member may extend between the lugs 20 to pass through the coupling member 190. The fixing member may be configured such that both ends thereof are coupled to the lugs 20. For example, both ends of the fixing member may be fitted into grooves formed in the lugs 20. In order to easily separate and couple the coupling member 190 from and to the lugs 20, the fixing member may be configured to be compressible in its longitudinal direction.

The coupling member 190 may include a first coupling member 190-1 and a second coupling member 190-2. The lugs 20 may include a first lug 21 coupled to the first coupling member 190-1 and a second lug 22 coupled to the second coupling member 190-2. Each of the first lug 21 and the second lug 22 may include a pair of protrusions extending from the side member 140. An end of the first coupling member 190-1 may be positioned between the pair of protrusions of the first lug 21. The first coupling member 190-1 may be connected to the housing 110 through the fixing member that passes through the first coupling member 190-1 and is coupled to the first lug 21. An end of the second coupling member 190-2 may be positioned between the pair of protrusions of the second lug 22. The second coupling member 190-2 may be connected to the housing 110 through the fixing member that passes through the second coupling member 190-2 and is coupled to the second lug 22.

The first coupling member 190-1 and the second coupling member 190-2 may be configured to be connected to or separable from each other. For example, a locking member fastening hole 193 may be formed in the first coupling member 190-1, and the second coupling member 190-2 may include a locking member 192, a band guide member 194, and a band fixing ring 195. The locking member 192 and the locking member fastening hole 193 are configured to be fastened to each other, so that the housing 110 and the coupling member 190 may be fixed to the user's body. The band guide member 194 may be configured to limit the range of movement of the locking member 192 when the locking member 192 is fastened to the locking member fastening hole 193, thereby making it possible for the coupling member 190 to be closely coupled to a part of the user's body. The band fixing ring 195 may limit the range of movement of the coupling member 190 in a state in which the locking member 192 and the locking member fastening hole 193 are fastened. By coupling or detaching the first coupling member 190-1 and the second coupling member 190-2 to or from each other, the electronic device 100 may be worn on the user's body (e.g., wrist) or detached from the body. However, the configuration of the coupling member 190 for maintaining the state of the electronic device 100 worn on the user's body is not limited to the above-described example, and various methods that are easy for those skilled in the art may be applied (e.g., folding clasp, fold-over clasp).

In one embodiment, the coupling member 190 may be formed in the form of a band or strap. The coupling member 190 may be at least partially made of an electrically conductive material (e.g., metal). The coupling member 190 at least partially including a conductive material may be referred to as a metal strap.

Although not shown, the electronic device 100 may further include a wheel key (not shown) that is disposed on the front surface 110A of the housing 110 and rotatable in at least one direction. For example, the wheel key may have a shape corresponding to the shape of the front member 120 (e.g., a circular frame). The wheel key may receive user input for implementing various functions of the electronic device 100 by being rotated by the user's manipulation. Optionally, the wheel key may be implemented in the form of a soft key on a touch-sensitive display device (e.g., the display 181 in FIG. 3) of the electronic device 100.

In one embodiment, the electronic device 100 may further include a connector hole (not shown) (e.g., a connecting terminal 1378 in FIG. 13). The connector hole may accommodate a connector (e.g., a USB connector) for transmitting and/or receiving electric power and/or data to and/or from an external electronic device (e.g., an electronic device 1302 in FIG. 13), or accommodate a connector (e.g., an earphone connector) for transmitting and/or receiving audio signals to and/or from the external electronic device.

In one embodiment, the electronic device 100 may further include another sensor (or sensor module) distinct from the first biometric sensor 10 and the second biometric sensor module 113, for example, at least one of a gesture sensor, a gyro sensor, an atmospheric pressure sensor, a magnetic sensor, an acceleration sensor, a grip sensor, a color sensor, an infrared ray (IR) sensor, a biometric sensor, a temperature sensor, a humidity sensor, or an illuminance sensor.

FIG. 3 is an exploded perspective view of the electronic device according to an embodiment of the disclosure. FIG. 3 may be a view in which a coupling member (e.g., the coupling member 190 in FIGS. 1 and 2) of the electronic device 100 is omitted. With respect to FIG. 3 and the following drawings, overlapping descriptions of components having the same reference numerals will be omitted.

Referring to FIG. 3, the electronic device 100 according to an embodiment may include the display 181, the bracket 182, the circuit board 150, and the battery 183.

In one embodiment, the display 181 may be disposed between the front member 120 and the bracket 182. At least a portion of the display 181 may be accommodated within an opening 141 of the side member 140. The display 181 may be attached to the front member 120. For example, the display 181 may be attached to a rear surface (e.g., a surface facing a second direction D2) of the front member 120. The display 181 may be visually exposed through the front member 120. For example, at least a portion of the display 181 may be visually exposed through the front member 120 in a front direction of the housing 110 (e.g., in a first direction D1).

The display 181 may be electrically connected to the circuit board 150. For example, the display 181 may be electrically connected to the circuit board 150 through a connector 185. The connector 185 may extend from the display 181 beyond the bracket 182 to the circuit board 150.

The display 181 may be combined with or disposed to be adjacent to a touch sensing circuit, a pressure sensor capable of measuring the intensity (pressure) of the touch, and/or a fingerprint sensor.

At least a portion of the front member 120 may be formed to be substantially transparent. The front member 120 may include a glass plate including various coating layers, or a polymer plate.

The front member 120 and the rear member 130 may be each coupled to the side member 140. The front member 120 may be coupled to the upper portion of the side member 140 (e.g., in the first direction D1), and the rear member 130 may be coupled to the lower portion of the side member 140 (e.g., in the second direction D2). As the front member 120, the rear member 130, and the side member 140 are coupled to each other, an internal space of the housing 110 may be formed. The display 181, the bracket 182, the circuit board 150, and/or the battery 183 may be at least partially accommodated in the internal space of the housing 110.

The side member 140 may surround the space between the front member 120 and the rear member 130. In one embodiment, the opening 141 through which the circuit board 150, the display 181, the bracket 182, or the battery 183 are at least partially accommodated may be formed in the side member 140. The front member 120 and the rear member 130 may be coupled to the side member 140 to face each other with the opening 141 therebetween.

At least a portion of the side member 140 may include a metal material and/or a polymer material. At least a portion of the side member 140 including a metal material may be configured as an antenna radiator for wireless communication.

The rear member 130 may be formed by coated or colored glass, ceramic, polymer, metal (e.g., aluminum, stainless steel (STS), or magnesium), or a combination of at least two of the above materials.

The bracket 182 may be disposed inside the housing 110, and may support other components of the electronic device 100 (e.g., the circuit board 150 and the battery 183). The bracket 182 may be disposed inside the opening 141 of the side member 140 to be surrounded by the side member 140. The bracket 182 may be formed of a metal material and/or a non-metal (e.g., polymer) material. The bracket 182 may be disposed between the rear member 130 and the display 181. The bracket 182 may provide a space (not shown) in which the battery 183 may be accommodated.

At least a portion of the housing 110 (e.g., the side member 140) may be made of metal. In this respect, the housing 110 may be referred to as a metal frame.

The battery 183 may supply electric power to at least some of the components of the electronic device 100. For example, the battery 183 may include, but is not limited to, a rechargeable secondary battery. The battery 183 may be disposed inside the housing 110 and supported by the bracket 182.

The circuit board 150 may be disposed between the rear member 130 and the bracket 182. The circuit board 150 may be supported by the bracket 182. The circuit board 150 may be mounted on the bracket 182 with its one surface (e.g., a surface facing the first direction D1). The circuit board 150 may be disposed so that the other side (e.g., the side facing the second direction D2) faces the rear member 130. The circuit board 150 may include, for example, a printed circuit board (PCB).

In one embodiment, on the circuit board 150, a processor (e.g., a processor 1320 in FIG. 13), a memory (e.g., a memory 1330 in FIG. 13), a communication module (e.g., a communication module 1390 in FIG. 13), a sensor module (e.g., a sensor module 1376 in FIG. 13), an interface (e.g., an interface 1377 in FIG. 13), and a connecting terminal (e.g., a connecting terminal 1378 in FIG. 13) may be disposed. The processor may include, for example, one or more of a central processing unit, an application processor, a graphic processing unit (GPU), a sensor processor, or a communication processor. The memory may include, for example, a volatile memory or a non-volatile memory. The interface may include, for example, a high-definition multimedia interface (HDMI), a universal serial bus (USB) interface, a secure digital (SD) card interface, or an audio interface. The interface may electrically or physically connect the electronic device 100 to an external electronic device, and may include a USB connector, an SD connector, a multimedia card (MMC) connector, or an audio connector.

Although not shown, the electronic device 100 may further include an antenna (not shown) (e.g., an antenna module 1397 in FIG. 13) provided in the form of a flat plate or a film. The antenna may perform communication with an external device, wirelessly transmit and receive electric power required for charging, and transmit a short-range communication signal or a magnetic-based signal including payment data. For example, the antenna may include a near field communication (NFC) antenna, a wireless charging antenna, or a magnetic secure transmission (MST) antenna. However, the above-described antenna is an embodiment, and is not limited thereto. For example, the electronic device 100 may be configured such that at least a portion of the housing 110 functions as an antenna. For example, an antenna structure may be formed by a portion of the side member 140 and/or the bracket 182 or a combination thereof.

FIG. 4A shows a state in which the electronic device is worn on the user's body according to an embodiment of the disclosure. FIG. 4B is a conceptual diagram showing a method of acquiring biometric information through the first biometric sensor according to an embodiment of the disclosure. In FIGS. 4A and 4B, the electronic device 100 is described based on a state in which the electronic device is worn on the user's left wrist (LW).

Referring to FIGS. 4A and 4B, the electronic device 100 according to an embodiment may be worn on the user's left wrist (LW). The rear surface 110B of the electronic device 100 may at least partially contact the left wrist (LW). The first electrode 11 and the second electrode 12 partially forming the rear surface 110B may be in contact with a first point B1 and a second point B2 of the left wrist (LW), respectively. A first finger (right finger (RF) 1) (e.g., the index finger) and a second finger (RF2) (e.g., the thumb) of the user's right hand may be in contact with the third electrode 13 and the fourth electrode 14, respectively.

A first electrical signal path P1 (indicated by a solid line in FIG. 4B) may be formed that leads through the first electrode 11, the first point B1 of the left wrist (LW), the first finger RF1 of the right hand, and the third electrode 13 with the user's body in contact with the first electrode 11 to the fourth electrode 14. A second electrical signal path P2 (indicated by a dotted line in FIG. 4B) may be formed that leads through the second electrode 12, the second point B2 of the left wrist (LW), the second finger RF2 of the right hand, and the fourth electrode 14 with the user's body in contact with the first electrode 11 to the fourth electrode 14. In FIG. 4B, the first electrical signal path P1 and the second electrical signal path P2 are shown outside the user's body, but this is for convenience of distinction, and the first electrical signal path P1 and the second electrical signal path P2 may include a user's upper body including both arms.

The first biometric sensor 10 may apply a current having a specified frequency (e.g., 50 kilohertz (KHz)) and a specified magnitude (e.g., 800 μA) to the first point B1 of the left wrist (LW) and the first finger RF1 of the right hand through the first electrode 11 and the third electrode 13. While the current is applied, the first biometric sensor 10 may detect a voltage between the second electrode 12 in contact with the second point B2 of the left wrist (LW) and the fourth electrode 14 in contact with the second finger RF2 of the right hand.

The electronic device 100 may determine a resistance value based on an applied current value and a measured voltage value. The electronic device 100 may acquire biometric information about the user's body water content and body fat content based on the determined resistance value. For example, the electronic device 100 may store data about height and weight previously entered by the user. The electronic device 100 may determine a body water content value and a body fat content value corresponding to the stored height and weight values and the determined resistance value from a pre-stored impedance index. However, the electronic device is not limited to the above example, and various bioelectric impedance analysis (BIA) methods may be applied. The operation of the electronic device 100 described above may be performed by a processor (e.g., processor 1320 in FIG. 13) that is operatively or electrically connected to the first biometric sensor 10.

In a comparative example, an unintended electrical signal path may be formed in addition to the first electrical signal path P1 and the second electrical signal path P2. For example, the current applied from the first electrode 11 to the left wrist (LW) may flow to the coupling member 190 in contact with the left wrist (LW). Then, the current may flow from the coupling member 190 to the housing 110 of the electronic device 100. The current flowing into the housing 110 may flow to a system ground of the electronic device 100, for example, a ground plane provided on a circuit board (e.g., the circuit board 150 in FIG. 3). Due to the unintended electrical signal path described above, the accuracy of biometric information detected from the first biometric sensor 10 may be reduced. For example, as the area where the coupling member 190 touches the user's body increases (for example, when the user tightly wears the electronic device 100), the measured body fat value may be lower than an actual value.

The electronic device 100 according to an embodiment may include an insulating structure configured to electrically separate the coupling member 190 and the housing 110 to prevent electrical signals applied from the electrodes from leaking into the housing 110 through the coupling member 190. The insulating structure of the electronic device 100 will be described with reference to the drawings below.

FIG. 5A is a view showing a fixing member according to an embodiment of the disclosure. FIG. 5B is a view showing a coupling member to which the fixing member is coupled, according to an embodiment of the disclosure. Reference numeral 501 in FIG. 5B is a view when a coupling member 190 is viewed along a longitudinal direction (e.g., a direction S1) of a fixing member 50. In FIG. 5B, a side member 140 and a lug 20 are shown as dotted lines.

Referring to FIG. 5A, the fixing member 50 according to an embodiment may include a body 51, a first pin 52, a second pin 53, an elastic member 54, and an insulating member 55. The body 51 may extend to have a longitudinal direction. The body 51 may have a cylindrical shape with a hollow formed therein. The first pin 52 and the second pin 53 may be disposed at both ends of the body 51. A portion of each of the first pin 52 and the second pin 53 may be accommodated within the hollow of the body 51, and the remaining portion may be positioned outside the hollow of the body 51. The elastic member 54 may be disposed within the hollow of the body 51. The elastic member 54 may be disposed between the first pin 52 and the second pin 53. The elastic member 54 may be configured to be compressed or tensioned along the longitudinal direction of the body 51. The elastic member 54 may provide, to the first pin 52 and the second pin 53, an elastic force in a direction corresponding to the longitudinal direction of the body 51 (e.g., a direction from the first pin 52 toward the second pin 53). The elastic member 54 may include, for example, a spring, but is not limited thereto. The first pin 52 and the second pin 53 may reciprocate within the hollow of the body 51 along the longitudinal direction. Flanges may be formed on the first pin 52 and the second pin 53 to limit the moving distance. The fixing member 50 may be compressed or tensioned as the first pin 52 and the second pin 53 move. The insulating member 55 may have a tube shape. The insulating member 55 may at least partially surround an outer peripheral surface of the body 51. The insulating member 55 may be made of an insulating material (e.g., an electrically insulating resin).

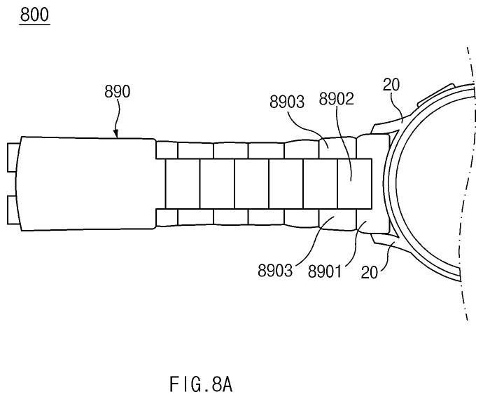

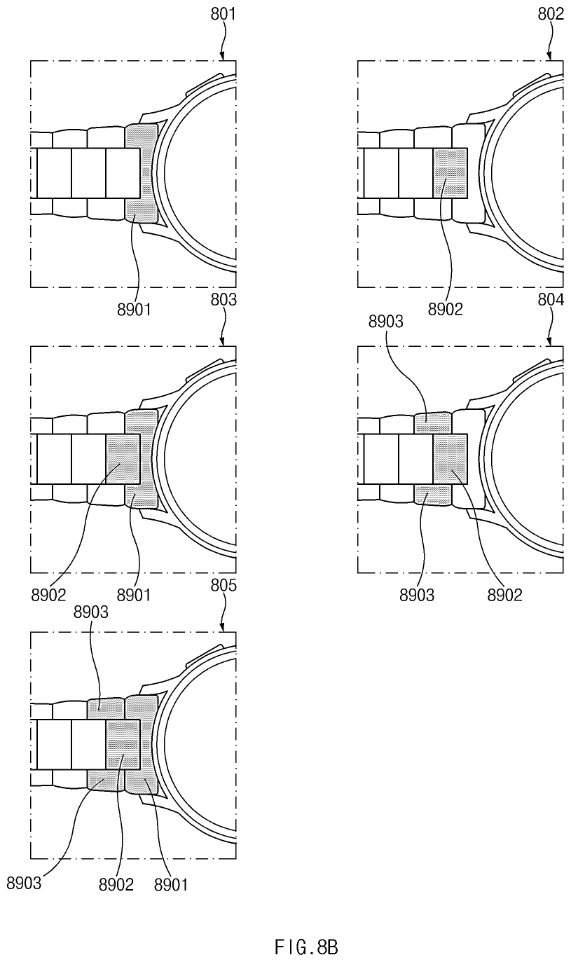

Referring to FIG. 5B, the coupling member 190 according to an embodiment may include a coupling portion 1901 connected to the lug 20. When the coupling member 190 is configured by assembling a plurality of links as shown in FIG. 5B, the coupling portion 1901 may be understood as including at least an end link (e.g., an end link 8901 in FIG. 8A) connected to the lug 20 among the plurality of links. For example, when the coupling member 190 includes an end link 190-1 and links 190-2, 190-3, and 190-4 connected to the end link 190-1, the coupling portion 1901 may include at least the end link 190-1.

A through hole 1902 in which the fixing member 50 is accommodated may be formed in the coupling portion 1901. The insulating member 55 of the fixing member 50 may be in contact with an inner peripheral surface of the through hole 1902. The first pin 52 and the second pin 53 of the fixing member 50 may be detachably connected to the lug 20.

The fixing member 50 may electrically separate the coupling member 190 from the housing (e.g., the side member 140) of the electronic device. The coupling portion 1901 of the coupling member 190 and the body 51 of the fixing member 50 may be electrically separated by the insulating member 55. In addition, the coupling member 190 may be electrically separated from the first pin 52 and the second pin 53 that are in contact with the body 51, and may be electrically separated from the lug 20 that is in contact with the first pin 52 and the second pin 53. Through the insulating member 55 of the fixing member 50, the unintended electrical signal path may be blocked and the accuracy of biometric information measurement may be improved.

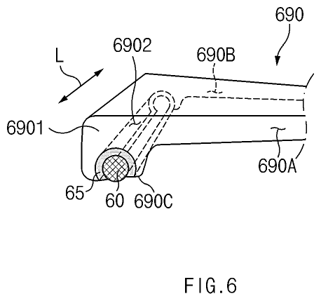

FIG. 6 is a view showing a coupling member to which a fixing member is coupled, according to an embodiment of the disclosure.

Referring to FIG. 6, an electronic device according to an embodiment may include a coupling member 690 and a fixing member 60. The description provided with reference to the coupling member 190 and the fixing member 50 may be applied to the coupling member 690 and the fixing member 60 in substantially the same, similar, or corresponding manner.

A groove 6902 may be formed in a coupling portion 6901 (e.g., the coupling portion 1901 in FIG. 5B) of the coupling member 690. The groove 6902 may be formed in one surface 690C of the coupling portion 6901. The one surface 690C may extend from a partial edge of a first side surface 690A of the coupling member 690 to a partial edge of a second side surface 690B opposite the first side surface 690A. When a user wears the electronic device, the one surface 690C may be a surface facing a user's body.

The groove 6902 may extend in a direction corresponding to a longitudinal direction of the fixing member 60 (e.g., the longitudinal direction L). The groove 6902 may extend from the first side surface 690A to the second side surface 690B of the coupling portion 6901. A cross-sectional shape of the groove 6902 cut perpendicular to the longitudinal direction L may partially correspond to a cross-sectional shape of the fixing member 60. For example, when the cross-sectional shape of the fixing member 60 is circular, the cross-sectional shape of the groove 6902 may be circular and open by a specified central angle. The specified central angle may be determined within a range in which the coupling member 690 is maintained in a state of being coupled to the fixing member 60, while the coupling member 690 is to be easily separated. For example, the specified central angle may be 30 degrees to 90 degrees, but is not limited thereto. Unlike the through hole 1902 in FIG. 5B that is formed in a cylindrical shape, the groove 6902 may be partially open through the one surface 690C of the coupling portion 6901. In this way, the fixing member 60 and the coupling member 690 may be easily coupled and separated.

An insulating member 65 may be disposed on an inner peripheral surface of the groove 6902. The insulating member 65 may be made of an electrically insulating material (e.g., rubber). The insulating member 65 may have a shape corresponding to the groove 6902. In one embodiment, the insulating member 65 may be formed along the inner peripheral surface of the groove 6902. In this case, the insulating member (e.g., the insulating member 55 in FIG. 5B) of the fixing member 60 may be omitted, but is not limited thereto. Optionally, unlike the fixing member 50, the fixing member 60 may not include the first pin 52, the second pin 53, and the elastic member 54. The fixing member 60 may be formed in a bar shape to be fixedly coupled to the lug 20.

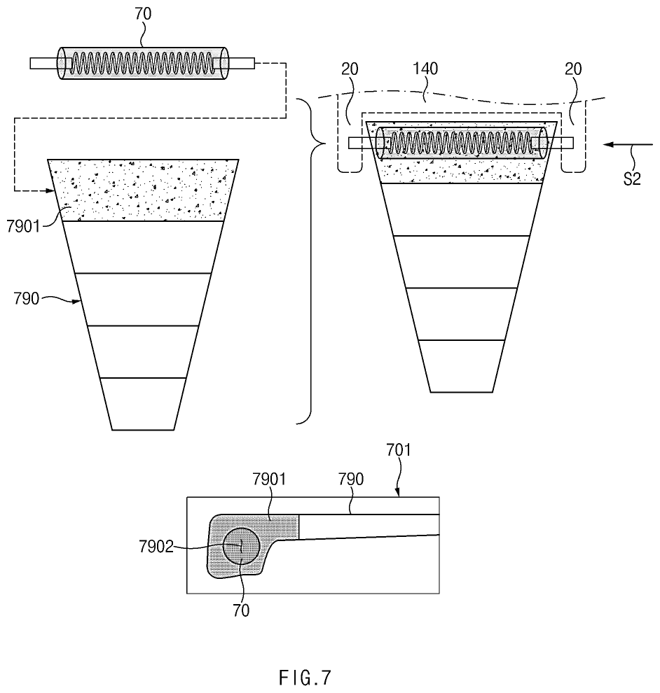

FIG. 7 is a view showing a coupling member to which a fixing member is coupled, according to an embodiment of the disclosure. Reference numeral 701 in FIG. 7 is a view when a coupling member 790 is viewed along a longitudinal direction (e.g., a direction S2) of a fixing member 70. In FIG. 7, a side member 140 and a lug 20 are shown as dotted lines.

Referring to FIG. 7, an electronic device according to an embodiment may include a coupling member 790 and a fixing member 70. The description provided with reference to the coupling member 190 or the coupling member 690 may be applied to the coupling member 790 in substantially the same, similar, or corresponding manner. The description provided with reference to the fixing member 50 or the fixing member 60 may be applied to the fixing member 70 in substantially the same, similar, or corresponding manner.

The coupling member 790 may include a coupling portion 7901 made of an electrically insulating material (e.g., resin, glass). A through hole 7902 (e.g., the through hole 1902 in FIG. 5B) may be formed in the coupling portion 7901. The coupling portion 7901 may be connected to the lug 20 through the fixing member 70 inserted into the through hole 7902.

When the coupling member 790 includes a plurality of interconnected links, the coupling portion 7901 may include at least a link (e.g., an end link 8901 in FIG. 8A) connected to the lug 20. The coupling member 790 may have a structure different from the above-described example. For example, the coupling member 790 may be configured in the form of a mesh band. In this case, the coupling member 790 may include a mesh-shaped metal band, and the coupling portion 7901 may be connected to an end of the metal band or may be coupled to cover the end of the metal band.

Because the coupling portion 7901 is made of an insulating material, the coupling member 790 may be electrically separated from the lug 20 and the side member 140. Because the coupling portion 7901 provides insulation, the fixing member 70 may not include an insulating member (e.g., the insulating member 55 in FIG. 5B). However, the insulation is not limited thereto, and the fixing member 70 may include the insulating member 55 in the same way as the fixing member 50 in FIG. 5A, and the insulating member 55, together with the coupling portion 7901, may provide electrical insulation between the coupling member 790 and the side member 140 and/or the lug 20.

FIG. 8A is a view showing a part of an electronic device according to an embodiment of the disclosure. FIG. 8B shows examples of insulating structures of the electronic device according to an embodiment of the disclosure.

Referring to FIG. 8A, an electronic device 800 (e.g., the electronic device 100 in FIG. 1) according to an embodiment may include a coupling member 890. The description provided with reference to the coupling member 790 in FIG. 7 may be applied to the coupling member 890 in substantially the same, similar, or corresponding manner.

The coupling member 890 may include a plurality of links. For example, the coupling member 890 may include an end link 8901 coupled to a lug 20, a center link 8902 connected to the end link 8901, and a side link 8903 connected to the center link 8902. The end link 8901, the center link 8902, and the side link 8903 may be coupled to be rotatable with each other.

In one embodiment, at least a portion of the plurality of links of the coupling member 890 may be made of an insulating material. For example, at least one of the plurality of links of the coupling member 890 may be made of an insulating material. For example, referring to FIG. 8B, the end link 8901, as indicated by reference numeral 801, may be made of an insulating material. For another example, as indicated by reference numeral 802, the center link 8902 may be made of an insulating material. In this case, in a state in which the user is wearing the electronic device, the center link 8902 may be in contact with a user's wrist, and the center link 8902 made of an insulating material may block an unintended electrical path, thereby improving the accuracy of biometric information acquisition. For another example, as indicated by reference numeral 803, the end link 8901 and the center link 8902 may be made of an insulating material. For still another example, as indicated by reference numeral 804, the center link 8902 and the side link 8903 may be made of an insulating material. For yet another example, as indicated by reference numeral 805, the end link 8901, the center link 8902, and the side link 8903 may be made of an insulating material.

The links made of an insulating material may prevent unintended electrical paths by insulating between the coupling member 890 and the housing of the electronic device.

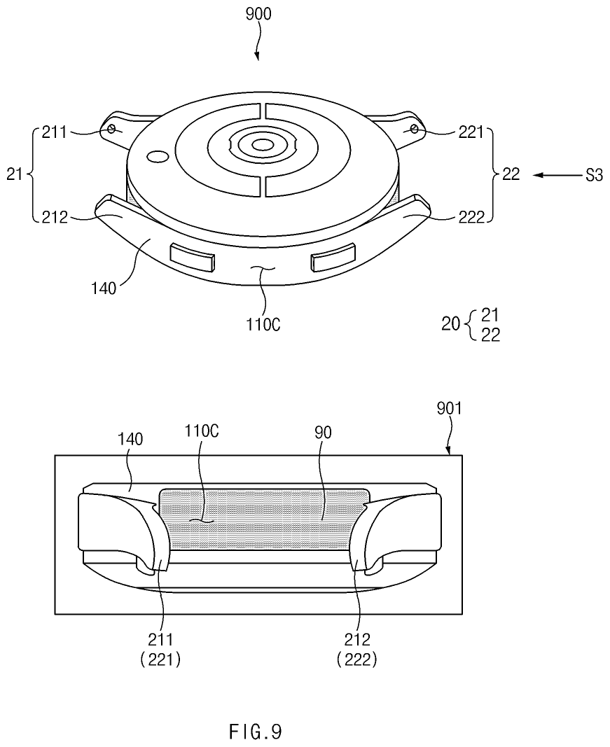

FIG. 9 shows a housing of an electronic device according to an embodiment of the disclosure. Reference numeral 901 in FIG. 9 is a view when a side surface 110C where a lug 20 of a side member 140 is positioned is viewed from the front (e.g., in a direction S3). Although a coupling member is omitted in FIG. 9, an electronic device 900 may include the above-described coupling members 190, 690, 790, and 890 and their fixing members 50, 60, and 70.

Referring to FIG. 9, the electronic device 900 (e.g., the electronic device 100 in FIG. 1 or the electronic device 800 in FIG. 8A) according to an embodiment may include an insulating layer 90. The insulating layer 90 may be at least partially formed on the side surface 110C of the electronic device 900. For example, the insulating layer 90 may be formed between protrusions of the lug 20. For example, the insulating layer 90 may be formed between a first protrusion 211 and a second protrusion 212 of a first lug 21. In addition, the insulating layer 90 may be formed between a third protrusion 221 and a fourth protrusion 222 of a second lug 22. The insulating layer 90 may be formed on the side surface 110C of the side member 140. The insulating layer 90 may be formed by coating or depositing an insulating material. The insulating layer 90 may electrically separate the coupling member (e.g., the coupling member 190 in FIG. 5B) and the side member 140. The insulating layer 90 may prevent the current applied to measure biometric information from flowing to the side member 140 through the coupling member that is in contact with a user's body.

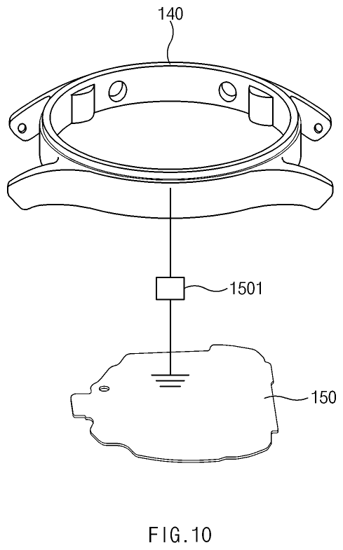

FIG. 10 shows a side member and a printed circuit board of an electronic device according to an embodiment of the disclosure. Although a coupling member is omitted in FIG. 10, an electronic device may include the above-described coupling members 190, 690, 790, and 890 and their fixing members 50, 60, and 70.

Referring to FIG. 10, a side member 140 according to an embodiment may be electrically connected to a printed circuit board 150 through an element 1501. For example, the side member 140 may be electrically connected to a ground portion provided on the printed circuit board 150 through the element 1501. The ground portion may include a layer of the printed circuit board 150 made of an electrically conductive material (e.g., copper). The element 1501 may be configured to block transmission of biological signals to the ground portion of the printed circuit board 150 through the side member 140. For example, the element 1501 may include at least one passive element, such as a capacitor, to block a biological signal with a specified frequency (e.g., 50 kHz). An element value of the capacitor may vary depending on the characteristics (e.g., frequency, intensity) of the current applied to measure biometric information. The element 1501 may be disposed on the printed circuit board 150. Components for electrically connecting the side member 140 and the printed circuit board 150 may include, for example, conductive traces provided by the printed circuit board 150 and a connector (e.g., C-clip) that electrically connects the conductive traces to the side member 140. However, the components are not limited thereto.

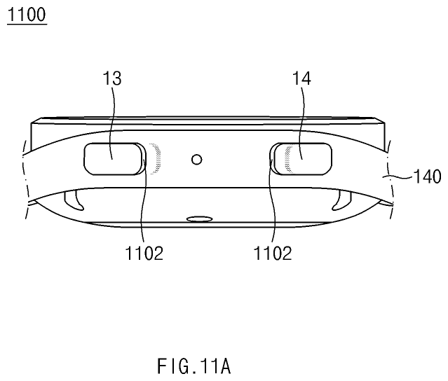

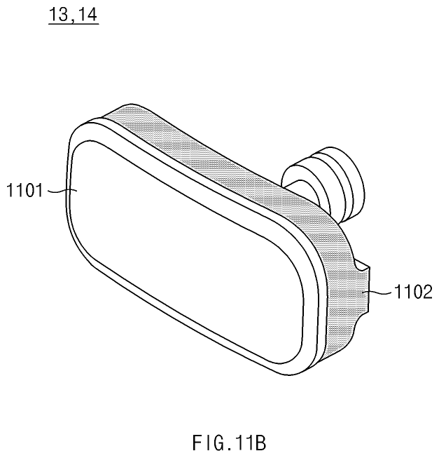

FIG. 11A shows an electronic device according to an embodiment of the disclosure. FIG. 11B shows electrodes of the electronic device according to an embodiment of the disclosure.

Referring to FIGS. 11A and 11B, a third electrode 13 and a fourth electrode 14 of an electronic device 1100 according to an embodiment may include a first portion 1101 and a second portion 1102. The first portion 1101 may be in contact with a user's body. The first portion 1101 may be made of an electrically conductive material (e.g., metal). The first portion 1101 may be spaced apart from a side member 140. The second portion 1102 may be connected to the first portion 1101. The second portion 1102 may be positioned between the first portion 1101 and the side member 140. The second portion 1102 may be at least partially inserted into the side member 140. The second portion 1102 may be made of an electrically insulating material (e.g., resin) to improve electrical insulation between the first portion 1101 and the side member 140.

Tables 1, 2, and 3 below show body impedance and body fat percentage measurement results of electronic devices according to the comparative example and an example of the disclosure. The electronic device according to the comparative example used a rubber strap made of an insulator.

TABLE 1

| | | Body | Body fat |
|---|---|---|---|
| First user | | impedance | percentage |
| Comparative example | Case 1 | 660.7 | 27 |
| | Case 2 | 663.4 | 27.2 |
| | Case 3 | 656.3 | 26.9 |
| | Average (A) | 660 | 27.0 |
| Example according | Case 1 | 652.2 | 26.8 |
| to the disclosure | Case 2 | 654.1 | 26.8 |
| | Case 3 | 663.6 | 27.2 |
| | Average (B) | 656 | 26.9 |

| | | |
|---|---|---|
| Relative error (B/A − 1) × 100% | −0.53% | −0.37% |

TABLE 2

| | | Body | Body fat |
|---|---|---|---|
| Second user | | impedance | percentage |
| Comparative example | Case 1 | 560 | 23 |
| | Case 2 | 551.7 | 22.6 |
| | Case 3 | 552.1 | 22.6 |
| | Average (A) | 555 | 22.7 |
| Example according | Case 1 | 541.4 | 22.1 |
| to the disclosure | Case 2 | 536.3 | 21.8 |
| | Case 3 | 535.5 | 21.7 |
| | Average (B) | 538 | 21.9 |

| | | |
|---|---|---|
| Relative error (B/A − 1) × 100% | −3.04% | −3.81% |

TABLE 3

| | | Body | Body fat |
|---|---|---|---|
| Third user | | impedance | percentage |
| Comparative example | Case 1 | 585 | 24.1 |
| | Case 2 | 582 | 24 |
| | Case 3 | 581 | 24 |
| | Average (A) | 583 | 24 |
| Example according | Case 1 | 583 | 24.1 |
| to the disclosure | Case 2 | 590 | 24.4 |
| | Case 3 | 586 | 24.2 |
| | Average (B) | 586 | 24.2 |

| | | |
|---|---|---|
| Relative error (B/A − 1) × 100% | 0.63% | 0.83% |

Referring to Table 1, Table 2, and Table 3, body composition measurement data of the electronic device according to the example of the disclosure and the electronic device according to the comparative example including the rubber strap show very similar results with a maximum relative error of 3.81% and a minimum relative error of 0.37%.

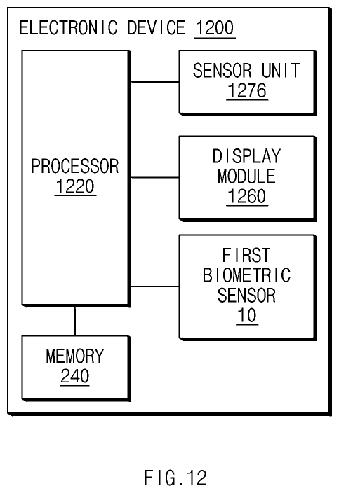

FIG. 12 is a block diagram of an electronic device according to an embodiment of the disclosure.

Referring to FIG. 12, an electronic device 1200 according to an embodiment may include a processor 1220, a memory 240, a sensor unit 1276, a display module 1260, and a first biometric sensor 10. In one embodiment, the processor 1220 (e.g., a processor 1320 in FIG. 13) may be operatively connected to the memory 240 (e.g., a memory 1330 in FIG. 13) to execute instructions stored in the memory 240. The processor 1220 may be operatively connected to the sensor unit 1276 (e.g., a sensor module 1376 in FIG. 13), the display module 1260 (e.g., a display module 1360 in FIG. 13), and the first biometric sensor 10, and control them by executing instructions stored in the memory 240. The processor 1220 may include at least one processor (e.g., an application processor) for controlling the sensor unit 1276, the display module 1260, and the first biometric sensor 10.

In one embodiment, the processor 1220 may determine whether a coupling member (e.g., the coupling member 190 in FIG. 1) is fastened to a user's body. For example, the processor 1220 may detect a position and/or orientation of the fastening member using the sensor unit 1276. The position and/or orientation of the fastening member may be determined, for example, based on the intensity and/or orientation of a magnetic field detected by the sensor unit 1276 (e.g., a geomagnetic sensor) through a magnet member installed on the coupling member (or a coupling member so that at least a portion thereof is magnetic), but are/is not limited thereto.

In one embodiment, the processor 1220 may display a user interface (UI) using the display module 1260 based on determining whether the coupling member is fastened to the user's body. For example, when it is determined that the coupling member is not fastened to the user's body, the processor 1220 may display the UI indicating a fastening state (e.g., not fastened) on the display module 1260. In addition, the processor 1220 may display, on the display module 1260, a visual object (e.g., “Press here if fastened”) instructing the user to fasten the coupling member.

When it is determined that the coupling member is fastened to the user's body and/or user input (e.g., touch input) to the visual object is received, the processor 1220 may display a UI requesting the user to input a reference value of biometric measurement. The reference value may include a BIA value measured through another electronic device or a BIA value measured through the electronic device 1200 using a strap other than a metal strap.

The processor 1220 may store the reference value of the biometric measurement input by the user in the memory 240.

The processor 1220 may perform the biometric information measurement operation a specified number of times (e.g., three or more times) using the first biometric sensor 10. The description provided with reference to FIGS. 4A and 4B may be equally applied to the biometric information measurement operation.

The processor 1220 may calculate an average value of the measured biometric information and acquire a correction value based on the calculated average value and the pre-stored reference value. For example, the processor 1220 may acquire a difference value between the reference value and the average value and determine the correction value by referring to a lookup table indicating a correction value corresponding to each difference value. The processor 1220 may correct the measured value of biometric information based on the correction value, and display the corrected biometric information value on the display module 1260.

A wearable electronic device (e.g., the electronic device 100 in FIG. 1) according to an embodiment may include a metal frame (e.g., the housing 110 in FIG. 1), a plurality of electrodes (e.g., the electrodes 11, 12, 12, and 14 in FIG. 4A) positioned on the metal frame to be in contact with a user's body and configured to form an electrical signal path with the user's body in contact to acquire biometric information, a metal strap (e.g., the coupling member 190 in FIG. 1) connected to the metal frame, and an insulating structure, and the insulating structure may be configured to electrically separate the metal strap and the metal frame so that the electrical signal does not leak to the metal frame through the metal strap.

The insulating structure may include a body (e.g., the body 51 in FIG. 5A) accommodated in a through hole (e.g., the through hole 1902 in FIG. 5B) formed in the metal strap, a first pin and a second pin disposed at both ends of the body and connected to the metal frame (e.g., the first pin 52 and the second pin 53 in FIG. 5B), and an insulating member (e.g., the insulating member 55 in FIG. 5B) surrounding the body so that the metal strap and the metal frame are electrically separated.

The insulating structure may include a body (e.g., the body 51 in FIG. 5A) accommodated in a groove (e.g., the groove 6902 in FIG. 6) formed in the metal strap, a first pin and a second pin disposed at both ends of the body and connected to the metal frame (e.g., the first pin 52 and the second pin 53 in FIG. 5A), and an insulating member (e.g., the insulating member 65 in FIG. 6) surrounding the body so that the metal strap and the metal frame are electrically separated.

The groove may extend from a first side surface of the metal strap (e.g., the first side surface 690A in FIG. 6) to a second side surface (e.g., the second side surface 690B in FIG. 6) opposite the first side surface in a longitudinal direction (e.g., the longitudinal direction L in FIG. 6), and the cross-sectional shape of the groove cut in a direction perpendicular to the longitudinal direction may have an open circular shape.

The metal strap may include a first surface (e.g., one surface 690C in FIG. 6) extending from a partial edge of the first side surface to a partial edge of the second side surface, and the first surface may face the user's body while the user is wearing the wearable electronic device, and the groove may be formed in the first surface.

The metal strap may include an end link (e.g., the coupling portion 7901 in FIG. 7) connected to the metal frame, and the end link may include an insulating material so that the metal strap and the metal frame are electrically separated.

The metal strap may include an end link (e.g., end link 8901 in FIGS. 8A and 8B) connected to the metal frame, a center link (e.g., the center link 8902 in FIGS. 8A and 8B) connected to the end link, and a side link (e.g., side link 8903 in FIGS. 8A and 8B) connected to the center link, and at least some of the end link, the center link, or the side link may be made of an insulating material.

At least one of the end link, the center link, or the side link may be made of an insulating material.

The wearable electronic device may include an insulating layer (e.g., the insulating layer 90 in FIG. 9), the metal frame may include a front surface, a rear surface opposite the front surface, and a side surface extending from the front surface to the rear surface (e.g., the side surface 110C in FIG. 9), and the insulating layer may be formed on at least a portion of the side surface.

The metal frame may include a first protrusion (e.g., the first protrusion 211 and/or the third protrusion 221 in FIG. 9) extending from the side surface and a second protrusion (e.g., the second protrusion 212 and/or the fourth protrusion 222 in FIG. 9), the metal strap may be connected between the first protrusion and the second protrusion, and the insulating layer may be formed in an area between the first protrusion and the second protrusion on the side surface.

The wearable electronic device may include a printed circuit board (e.g., the printed circuit board 150 in FIG. 10) and include at least one element (e.g., the element 1501 in FIG. 10) disposed on the printed circuit board and electrically connected to a ground portion of the printed circuit board, and the metal frame may be electrically connected to the at least one element.

The at least one element may include a capacitor.

The plurality of electrodes may include a first portion made of an electrically conductive material (e.g., the first portion 1101 in FIG. 11B) and a second portion formed of an electrically insulating material (e.g., the second portion 1102 in FIG. 11B).

The first portion may be spaced apart from the metal frame, and may be configured to form an electrical signal path with a user's body in contact.

The second portion may be connected to the first portion, may be at least partially accommodated in the metal frame, and may be configured to electrically insulate the first portion and the metal frame.

A method of acquiring the biometric information by the wearable electronic device may include determining whether the metal strap is fastened to a user, detecting the biometric information based on the determining, correcting the detected biometric information based on a pre-stored reference value of biometric information, and displaying the corrected biometric information.

The method may include detecting the biometric information in response to determining that the metal strap is fastened to the user.

The method may include displaying a visual object requesting the user to fasten the metal strap, in response to determining that the metal strap is not fastened to the user.

The method may include receiving user input for the visual object and detecting the biometric information in response to receiving the user input.

The method may include displaying a user interface requesting input of the reference value.

FIG. 13 is a block diagram showing an electronic device 1301 in a network environment 1300 according to an embodiment of the disclosure.

Referring to FIG. 13, the electronic device 1301 in the network environment 1300 may communicate with an electronic device 1302 via a first network 1398 (e.g., a short-range wireless communication network), or communicate with at least one of an electronic device 1304 or a server 1308 via a second network 1399 (e.g., a long-range wireless communication network). According to an embodiment, the electronic device 1301 may communicate with the electronic device 1304 via the server 1308. According to an embodiment, the electronic device 1301 may include a processor 1320, a memory 1330, an input module 1350, a sound output module 1355, a display module 1360, an audio module 1370, a sensor module 1376, an interface 1377, a connecting terminal 1378, a haptic module 1379, a camera module 1380, a power management module 1388, a battery 1389, a communication module 1390, a subscriber identification module (SIM) 1396, or an antenna module 1397. In some embodiments, at least one (e.g., the connecting terminal 1378) of the components may be omitted from the electronic device 1301, or one or more other components may be added in the electronic device 1301. In some embodiments, some (e.g., the sensor module 1376, the camera module 1380, or the antenna module 1397) of the components may be integrated into one component (e.g., the display module 1360).

The processor 1320 may execute, for example, software (e.g., a program 1340) to control at least one other component (e.g., a hardware or software component) of the electronic device 1301 coupled to the processor 1320, and may perform various data processing or computation. According to an embodiment, as at least part of the data processing or computation, the processor 1320 may store a command or data received from another component (e.g., the sensor module 1376 or the communication module 1390) in a volatile memory 1332, process the command or the data stored in the volatile memory 1332, and store resulting data in a non-volatile memory 1334. According to an embodiment, the processor 1320 may include a main processor 1321 (e.g., a central processing unit (CPU) or an application processor (AP)), or an auxiliary processor 1323 (e.g., a graphics processing unit (GPU), a neural processing unit (NPU), an image signal processor (ISP), a sensor hub processor, or a communication processor (CP)) that is operable independently from, or in conjunction with the main processor 1321. For example, when the electronic device 1301 includes the main processor 1321 and the auxiliary processor 1323, the auxiliary processor 1323 may be configured to use less power than the main processor 1321 or to be specialized for a specified function. The auxiliary processor 1323 may be implemented as separate from, or as part of the main processor 1321.

The auxiliary processor 1323 may control at least some of functions or states related to at least one component (e.g., the display module 1360, the sensor module 1376, or the communication module 1390) among the components of the electronic device 1301, instead of the main processor 1321 while the main processor 1321 is in an inactive (e.g., sleep) state, or together with the main processor 1321 while the main processor 1321 is in an active state (e.g., executing an application), for example. According to an embodiment, an auxiliary processor 1323 (e.g., an image signal processor or a communication processor) may be provided as part of another component (e.g., the camera module 1380 or the communication module 1390) functionally related to the auxiliary processor. According to an embodiment, the auxiliary processor 1323 (e.g., a neural network processing device) may include a hardware structure specialized for processing an artificial intelligence model. The artificial intelligence model may be generated through machine learning. Such learning may be performed, for example, in the electronic device 1301 itself on which an artificial intelligence model is performed, or may be performed through a separate server (e.g., the server 1308). The learning algorithm may include, for example, supervised learning, unsupervised learning, semi-supervised learning, or reinforcement learning, but is not limited to the above-mentioned examples. The artificial intelligence model may include a plurality of artificial neural network layers. The artificial neural network may be one of a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-networks, or a combination of two or more of the above networks, but is not limited to the above examples. The artificial intelligence model may additionally or alternatively include a software structure, in addition to the hardware structure.

The memory 1330 may store various data to be used by at least one component (e.g., the processor 1320 or the sensor module 1376) of the electronic device 1301. The various data may include, for example, software (e.g., the program 1340) and input data or output data for a command related thereto. The memory 1330 may include the volatile memory 1332 or the non-volatile memory 1334.

The program 1340 may be stored in the memory 1330 as software, and may include, for example, an operating system (OS) 1342, middleware 1344, or an application 1346.

The input module 1350 may receive a command or data to be used by a component (e.g., the processor 1320) of the electronic device 1301, from the outside (e.g., the user) of the electronic device 1301. The input module 1350 may include, for example, a microphone, a mouse, a keyboard, a key (e.g., a button), or a digital pen (e.g., a stylus pen).

The sound output module 1355 may output sound signals to the outside of the electronic device 1301. The sound output module 1355 may include, for example, a speaker or a receiver. The speaker may be used for general purposes, such as playing multimedia or playing record. The receiver may be used for receiving incoming calls. According to an embodiment, the receiver may be implemented separately from the speaker, or as part thereof.

The display module 1360 may visually provide information to the outside (e.g., the user) of the electronic device 1301. The display module 1360 may include, for example, a display, a hologram device, or a projector and control circuitry to control a corresponding one of the display, hologram device, and projector. According to an embodiment, the display module 1360 may include a touch sensor adapted to detect a touch, or a pressure sensor adapted to measure the strength of force incurred by the touch.

The audio module 1370 may convert sound into an electrical signal and vice versa. According to an embodiment, the audio module 1370 may acquire the sound via the input module 1350, or may output the sound via the sound output module 1355 or an external electronic device (e.g., the electronic device 1302) (e.g., a speaker or a headphone) directly or wirelessly connected to the electronic device 1301.

The sensor module 1376 may detect an operational state (e.g., power or temperature) of the electronic device 1301 or an external environmental state (e.g., a state of the user), and then generate an electrical signal or data value corresponding to the detected state. According to an embodiment, the sensor module 1376 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 1377 may support one or more specified protocols to be used for the electronic device 1301 to be connected to an external electronic device (e.g., the electronic device 1302) directly or wirelessly. According to an embodiment, the interface 1377 may include, for example, a high definition multimedia interface (HDMI), a universal serial bus (USB) interface, a secure digital (SD) card interface, or an audio interface.

The connecting terminal 1378 may include a connector via which the electronic device 1301 may be physically connected to the external electronic device (e.g., the electronic device 1302). According to an embodiment, the connecting terminal 1378 may include, for example, an HDMI connector, a USB connector, an SD card connector, or an audio connector (e.g., a headphone connector).

The haptic module 1379 may convert an electrical signal into a mechanical stimulus (e.g., a vibration or a movement) or electrical stimulus which may be recognized by a user via his/her tactile sensation or kinesthetic sensation. According to an embodiment, the haptic module 1379 may include, for example, a motor, a piezoelectric element, or an electric stimulator.

The camera module 1380 may capture a still image or moving images. According to an embodiment, the camera module 1380 may include one or more lenses, image sensors, image signal processors, or flashes.

The power management module 1388 may manage power supplied to the electronic device 1301. According to an embodiment, the power management module 1388 may be implemented as at least part of, for example, a power management integrated circuit (PMIC).

The battery 1389 may supply power to at least one component of the electronic device 1301. According to an embodiment, the battery 1389 may include, for example, a primary cell which is not rechargeable, a secondary cell which is rechargeable, or a fuel cell.

The communication module 1390 may support establishing a direct (e.g., wired) communication channel or a wireless communication channel between the electronic device 1301 and the external electronic device (e.g., the electronic device 1302, the electronic device 1304, or the server 1308) and performing communication via the established communication channel. The communication module 1390 may include one or more communication processors that are operable independently from the processor 1320 (e.g., the application processor (AP)) and support a direct (e.g., wired) communication or a wireless communication. According to an embodiment, the communication module 1390 may include a wireless communication module 1392 (e.g., a cellular communication module, a short-range wireless communication module, or a global navigation satellite system (GNSS) communication module) or a wired communication module 1394 (e.g., a local area network (LAN) communication module or a power line communication (PLC) module). A corresponding one of these communication modules may communicate with the external electronic device 1304 via the first network 1398 (e.g., a short-range communication network, such as Bluetooth™, wireless-fidelity (Wi-Fi) direct, or infrared data association (IrDA)) or the second network 1399 (e.g., a long-range communication network, such as a legacy cellular network, a fifth generation (5G) network, a next-generation communication network, the Internet, or a computer network (e.g., LAN or wide area network (WAN)). These various types of communication modules may be integrated into a single component (e.g., a single chip), or may be implemented as multi components (e.g., multi chips) separate from each other. The wireless communication module 1392 may identify or authenticate the electronic device 1301 in a communication network, such as the first network 1398 or the second network 1399, using subscriber information (e.g., international mobile subscriber identity (IMSI)) stored in the subscriber identification module 1396.

The wireless communication module 1392 may support a 5G network after a fourth generation (4G) network and a next-generation communication technology, for example, a new radio (NR) access technology. NR access technology may support a high-speed transmission of high-capacity data (enhanced mobile broadband (eMBB)), minimization of terminal power and access to multiple terminals (massive machine type communications (mMTC)), or high reliability and low latency (ultra-reliable and low-latency communications (URLLC)). The wireless communication module 1392 may support a high frequency band (e.g., millimeter wave (mmWave) band) to achieve a high data rate, for example. The wireless communication module 1392 may support various techniques for securing performance in a high frequency band, for example, beamforming, massive multiple-input and multiple-output (MIMO), full dimensional MIMO (FD-MIMO), array antenna, analog beamforming, or large scale antenna. The wireless communication module 1392 may support various requirements defined in the electronic device 1301, an external electronic device (e.g., the electronic device 1304), or a network system (e.g., the second network 1399). According to an embodiment, the wireless communication module 1392 may support a peak data rate (e.g., 20 gigabits per second (Gbps) or more) for achieving the eMBB, loss coverage (e.g., 164 decibels (dB) or less) for achieving the mMTC, or U-plane latency (e.g., 0.5 milliseconds (ms) or less each for downlink (DL) and uplink (UL), or 1 ms or less for the round trip) for achieving the URLLC.

The antenna module 1397 may transmit or receive a signal or power to or from the outside (e.g., an external electronic device). According to an embodiment, the antenna module 1397 may include an antenna including a radiating element composed of a conductive material or a conductive pattern formed on a substrate (e.g., PCB). According to an embodiment, the antenna module 1397 may include a plurality of antennas (e.g., an antenna array). In such a case, at least one antenna appropriate for a communication scheme used in the communication network, such as the first network 1398 or the second network 1399, may be selected from among the plurality of antennas, for example, by the communication module 1390. The signal or the power may be transmitted or received between the communication module 1390 and the external electronic device via the selected at least one antenna. According to some embodiments, another component (e.g., a radio frequency integrated circuit (RFIC)) other than the radiating element may be additionally formed as a part of the antenna module 1397.

According to various embodiments, the antenna module 1397 may form an mmWave antenna module. According to an embodiment, the mmWave antenna module may include a printed circuit board, an RFIC disposed on or adjacent to a first surface (e.g., a bottom surface) of the printed circuit board and capable of supporting a specified high frequency band (e.g., mmWave band), and a plurality of antennas (e.g., an array antenna) disposed on or adjacent to a second surface (e.g., top or side surface) of the printed circuit board and capable of transmitting or receiving signals of the designated high frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 1301 and the external electronic device 1304 via the server 1308 coupled with the second network 1399. Each of the external electronic devices 1302 and 1304 may be a device of a same type as, or a different type, from the electronic device 1301. According to an embodiment, all or some of operations to be executed at the electronic device 1301 may be executed at one or more of the external electronic devices 1302, 1304, and 1308. For example, if the electronic device 1301 should perform a function or a service automatically, or in response to a request from a user or another device, the electronic device 1301, instead of, or in addition to, executing the function or the service by itself, may request one or more external electronic devices to perform at least part of the function or the service. The one or more external electronic devices receiving the request may perform the at least part of the function or the service requested, or an additional function or an additional service related to the request, and transfer an outcome of the performing to the electronic device 1301. The electronic device 1301 may provide the outcome, with or without further processing of the outcome, as at least part of a reply to the request. To this end, a cloud computing, distributed computing, mobile edge computing (MEC), or client-server computing technology may be used, for example. The electronic device 1301 may provide an ultra-low latency service using, for example, distributed computing or mobile edge computing. In another embodiment, the external electronic device 1304 may include an Internet of things (IoT) device. The server 1308 may be an intelligent server using machine learning and/or neural networks. According to an embodiment, the external electronic device 1304 or the server 1308 may be included in the second network 1399. The electronic device 1301 may be applied to an intelligent service (e.g., smart home, smart city, smart car, or health care) based on 5G communication technology and IoT-related technology.

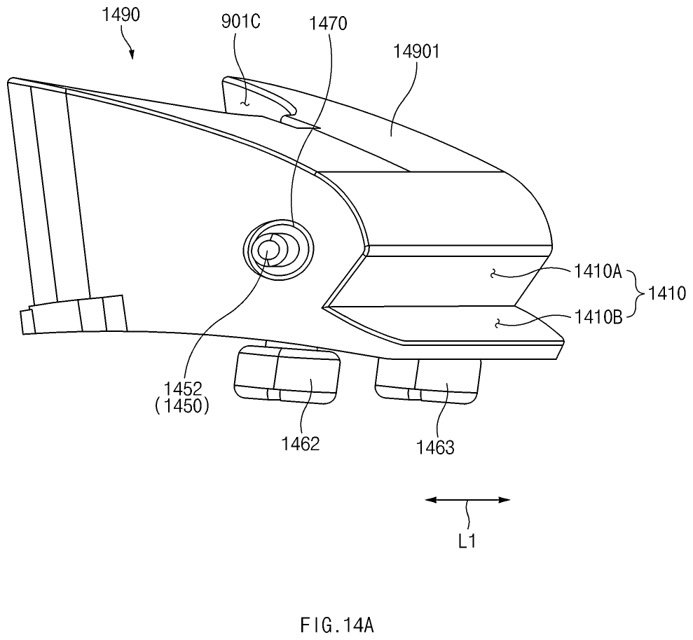

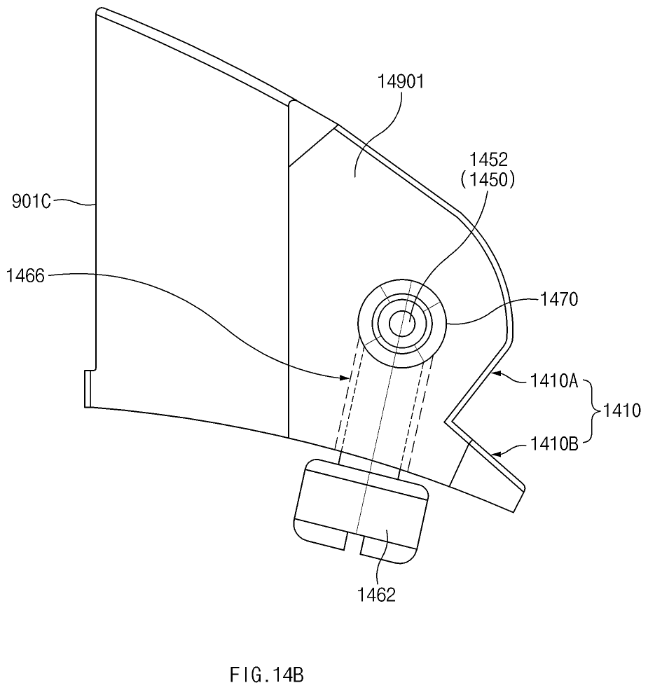

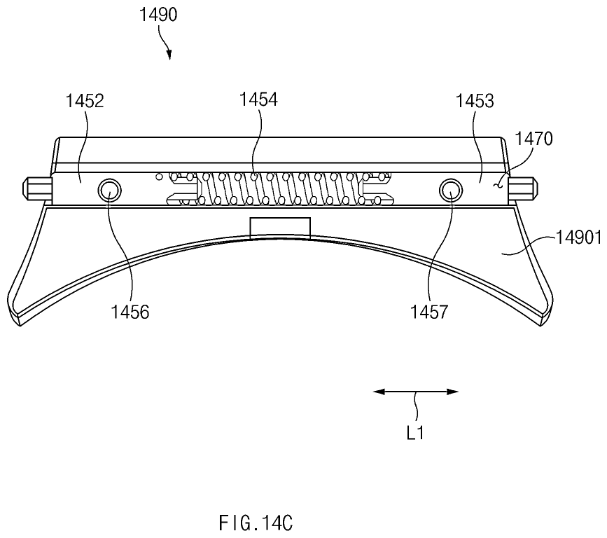

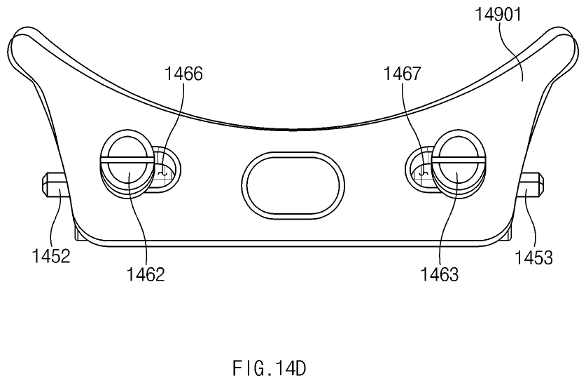

FIG. 14A is a view showing a coupling member according to an embodiment of the disclosure. FIG. 14B is a view showing the coupling member according to an embodiment of the disclosure. FIG. 14C is a view showing the coupling member according to an embodiment of the disclosure. FIG. 14C may be a cross-section of a first fixing member 1450 in FIG. 14A cut in a longitudinal direction L1. FIG. 14D is a view showing the coupling member according to an embodiment of the disclosure.

Referring to FIGS. 14A, 14B, 14C, and 14D, a coupling member 1490 according to an embodiment may include an end link 14901, a first fixing member 1450, and a second fixing member 1462, and a third fixing member 1463. The description provided with reference to the coupling member 190 in FIG. 1, the coupling member 190 in FIGS. 5A and 5B, the coupling member 690 in FIG. 6, the coupling member 790 in FIG. 7, and the coupling member 890 in FIGS. 8A and 8B may be applied to the coupling member 1490 in substantially the same, similar, or corresponding manner. The description provided with reference to the coupling portion 1901 in FIGS. 5A and 5B, the coupling portion 6901 in FIG. 6, the coupling portion 7901 in FIG. 7, and the end link 8901 in FIGS. 8A and 8B may be applied to the end link 14901 in substantially the same, similar, or corresponding manner.

In an embodiment, the end link 14901 may be detachably connected to a lug (e.g., the lug 20 in FIG. 9) of the electronic device. The end link 14901 may include a side surface 901C. The side surface 901C may face the housing (e.g., the side member 140 in FIG. 9) of the electronic device. For example, the side surface 901C may face a side surface (e.g., the side surface 110C in FIG. 9) of the housing. For example, the side surface 901C may face an insulating layer (e.g., the insulating layer 90 in FIG. 9) formed on the side surface of the electronic device. The side surface 901C may have a shape corresponding to the side surface of the housing. The side surface 901C may, for example, be at least partially in contact with the side surface of the housing, but is not limited thereto.

In one embodiment, the first fixing member 1450 may be disposed within a through hole 1470 formed in the end link 14901. The first fixing member 1450 may include a first pin 1452, a second pin 1453, and an elastic member 1454. The first pin 1452 and the second pin 1453 may be configured to be movable within the through hole 1470 along the longitudinal direction L1 of the through hole 1470. The first pin 1452 and the second pin 1453 may include a portion that protrudes out of the through hole 1470 to be coupled to the lug. The elastic member 1454 may be compressed or tensioned between the first pin 1452 and the second pin 1453. The description of the fixing member 50 in FIGS. 5A and 5B may be applied to the first fixing member 1450 in substantially the same, similar, or corresponding manner. For example, the first fixing member 1450 may include an insulating member (e.g., the insulating member 55 in FIG. 5B) that accommodates the first pin 1452, the second pin 1453, and the elastic member 1454, but is not limited thereto.

In one embodiment, a second fixing member 1462 may be at least partially disposed in a through hole 1466 formed in the end link 14901. The through hole 1466 may be formed in a direction substantially perpendicular to the through hole 1470. The through hole 1466 may extend from one surface of the end link 14901 to the first pin 1452. The through hole 1466 may extend along a longitudinal direction of the first pin 1452 to provide a space in which the second fixing member 1462 is movable. The second fixing member 1462 may pass through the through hole 1466 and be fastened to a groove 1456 formed in the first pin 1452. The third fixing member 1463 may be at least partially disposed in a through hole 1467 formed in the end link 14901. The through hole 1467 may be formed in the direction substantially perpendicular to the through hole 1470. The through hole 1467 may extend from one surface of the end link 14901 to the second pin 1453. The through hole 1467 may extend along a longitudinal direction of the second pin 1453 to provide a space in which the third fixing member 1463 is movable. The third fixing member 1463 may pass through the through hole 1467 and be fastened to a groove 1457 formed in the second pin 1453. The second fixing member 1462 may include, for example, a first screw, but is not limited thereto. For example, the first screw may be fastened to a thread formed in the through hole 1466 and/or the groove 1456, but is not limited thereto. The third fixing member 1463 may include, for example, a second screw, but is not limited thereto. The second screw may be fastened to a thread formed in the through hole 1467 and/or the groove 1457, but is not limited thereto. In one embodiment, the second fixing member 1462 and the third fixing member 1463 may be configured to be caught on the periphery of the through hole 1466 and the through hole 1467 as the second fixing member 1462 and the third fixing member 1463 are fastened to the through hole 1466 and the through hole 1467. For example, the second fixing member 1462 and the third fixing member 1463 may include a portion (e.g., a head of a screw) formed larger than diameters of the through hole 1466 and the through hole 1467 so that it cannot pass through the through hole 1466 and the through hole 1467, but are not limited thereto. The completely fastened second fixing member 1462 and third fixing member 1463 may be fixed on the surface of the end link 14901. In one embodiment, depending on a degree to which the second fixing member 1462 and the third fixing member 1463 are fastened to the groove 1456 and the groove 1457, the second fixing member 1462 and the third fixing member 1463 may be restricted in movement, or may move along the through hole 1466 and the through hole 1467. Accordingly, the first pin 1452 and the second pin 1453 connected to the second fixing member 1462 and the third fixing member 1463 may be restricted in movement, or may move with the movement of the second fixing member 1462 and the third fixing member 1463. The user may easily couple the first pin 1452 and the second pin 1453 to the lug or easily separate them from the lug by manipulating the second fixing member 1462 and the third fixing member 1463.

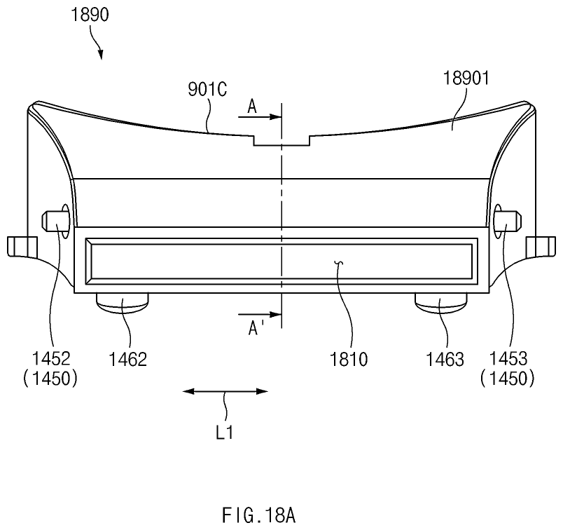

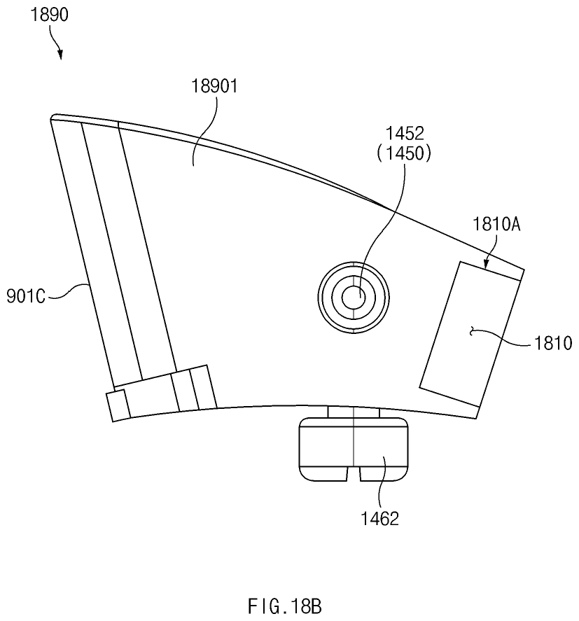

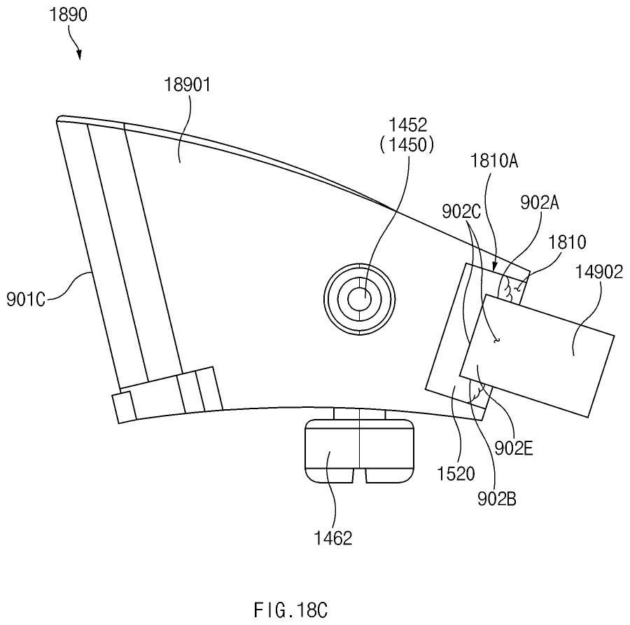

In one embodiment, a coupling groove 1410 may be formed in the end link 14901. The coupling groove 1410 may be formed in one surface of the end link 14901. For example, the coupling groove 1410 may be depressed toward the side surface 901C in a surface opposite the side surface 901C. The coupling groove 1410 may be defined by a first surface 1410A and a second surface 1410B. The first surface 1410A and the second surface 1410B may extend from the one surface toward the side surface 901C. At a position closer to the side surface 901C, a distance between the first surface 1410A and the second surface 1410B may get shorter. A depth of the coupling groove 1410 may be defined by an edge of the first surface 1410A and an edge of the second surface 1410B meeting each other. Both ends of the coupling groove 1410 may be open. For example, both ends of the coupling groove 1410 corresponding to the longitudinal direction L1 of the through hole 1470 may be open, but are not limited thereto. Alternatively, the coupling groove 1410 may be provided with both ends closed, like a coupling groove 1457 of FIG. 18A. The coupling groove 1410 may be referred to as a coupling surface 1410.

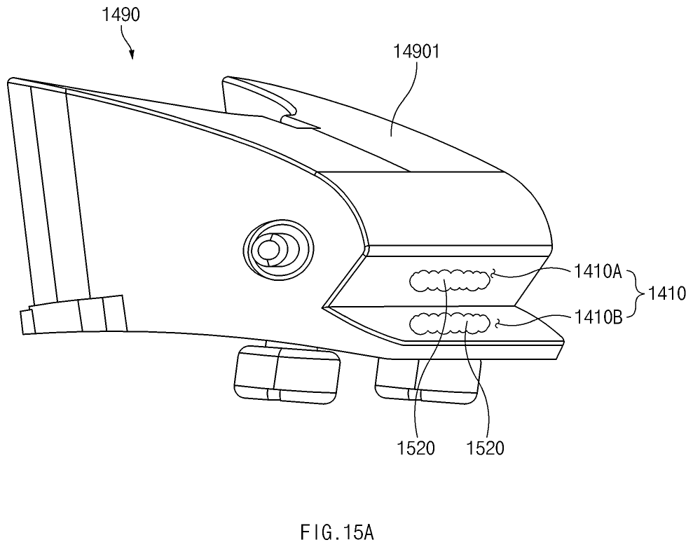

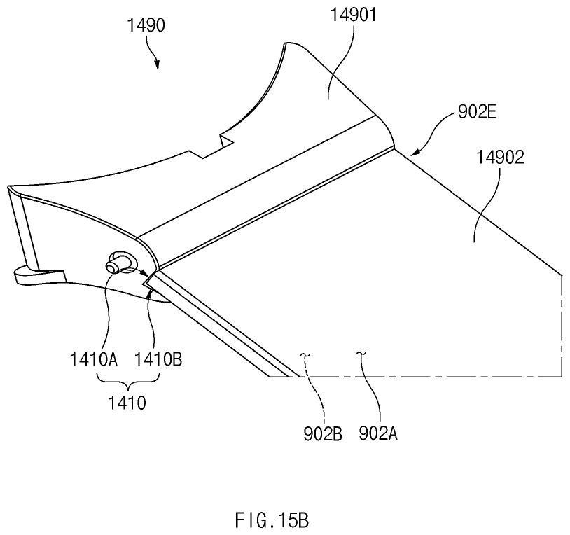

FIG. 15A is a view showing an end link to which an adhesive member is applied, according to an embodiment of the disclosure. FIG. 15B is a view showing a coupling member in which a strap is coupled to the end link, according to an embodiment of the disclosure.

Referring to FIGS. 15A and 15Bb, a coupling member 1490 according to an embodiment may include a strap 14902. The strap 14902 may include an end 902E, a first surface 902A, and a second surface 902B opposite the first surface 902A. In one embodiment, the strap 14902 may be made at least in part of a conductive material (e.g., metal). The strap 14902 may be formed, for example, in a mesh shape, but is not limited thereto. For example, the strap 14902 may include a plurality of link members rotatably connected to each other (e.g., the center link 8902 and the side link 8903 in FIG. 8A).

In one embodiment, the end 902E of the strap 14902 may be accommodated in a coupling groove 1410 of the end link 14901. The coupling groove 1410 may surround at least the first surface 902A and the second surface 902B of the end 902E.

In one embodiment, the strap 14902 may be coupled to the end link 14901. For example, the end 902E of the strap 14902 may be coupled to the end link 14901. An adhesive member 1520 may be applied within the coupling groove 1410 of the end link 14901. The adhesive member 1520 may be determined considering flowability, adhesive strength, electrical conductivity, resistance to environmental changes, and the like. The adhesive member 1520 may be made of a non-conductive material. For example, the adhesive member 1520 may include an adhesive made of epoxy-based resin. The end 902E of the strap 14902 may be inserted into the coupling groove 1410 to which the adhesive member 1520 is applied. To improve adhesion, a heat curing process for the adhesive member 1520 may be performed after insertion of the strap 14902. The heat curing process may be performed, for example, at 150 degrees Celsius for 30 minutes, but is not limited thereto. After the heat curing process, a joint strength of the end link 14901 and the strap 14902 may be about 122.11 kgf. This may be more than six times higher than 20 kgf, which is the general management standard for joint strength when joined through a welding process.

In one embodiment, the adhesive member 1520 may at least partially surround the end 902E of the strap 14902. The adhesive member 1520 may be at least partially disposed on a first surface 1410A of the coupling groove 1410 and/or at least partially disposed on a second surface 1410B thereof. The adhesive member 1520 may be at least partially disposed between the end 902E of the strap 14902 and the coupling groove 1410. For example, the adhesive member 1520 may be at least partially disposed between the end 902E of the strap 14902 and the first surface 1410A of the coupling groove 1410, and/or at least partially disposed between the end 902E of the strap 14902 and the second surface 1410B of the coupling groove 1410.

In one embodiment, the strap 14902 may be spaced apart from the end link 14901. For example, the end 902E of the strap 14902 may be spaced apart from the first surface 1410A and the second surface 1410B of the coupling groove 1410. The strap 14902 may be spaced apart from the end link 14901 through the adhesive member 1520. The strap 14902 may be physically and electrically separated from the end link 14901. The strap 14902 may be electrically separated from a housing of the electronic device (e.g., the housing 110 in FIG. 1) through the adhesive member 1520. In this way, unintended electrical paths may be blocked and the accuracy of biometric information acquisition may be improved. An insulating structure of the electronic device according to an embodiment may include the adhesive member 1520 that joins the strap 14902 and the end link 14901.

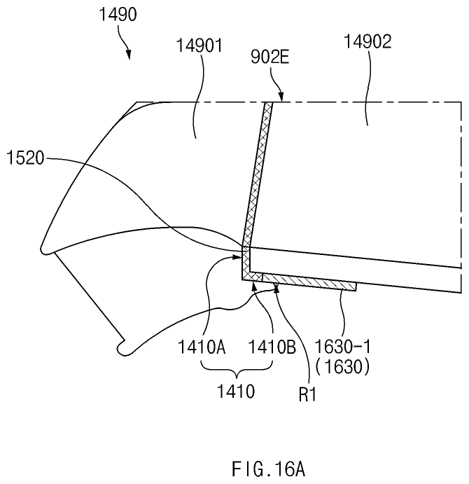

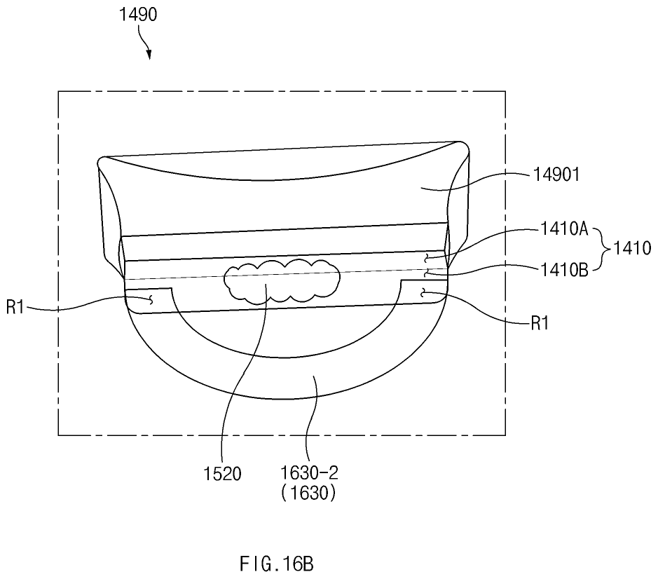

FIG. 16A is a view showing a coupling member including a blocking member according to an embodiment of the disclosure. FIG. 16B is a view showing an example of the blocking member according to an embodiment.

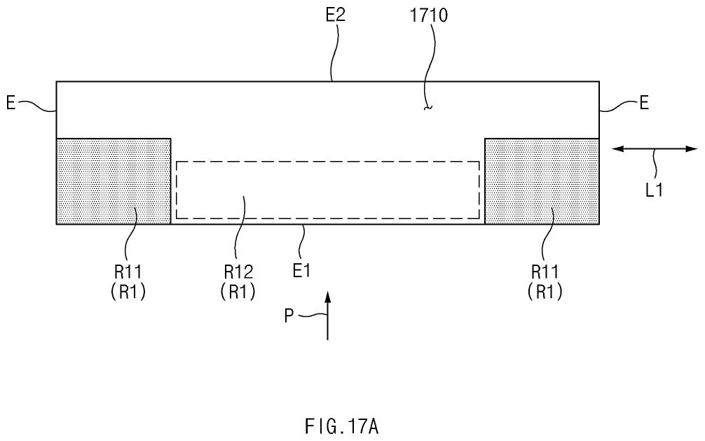

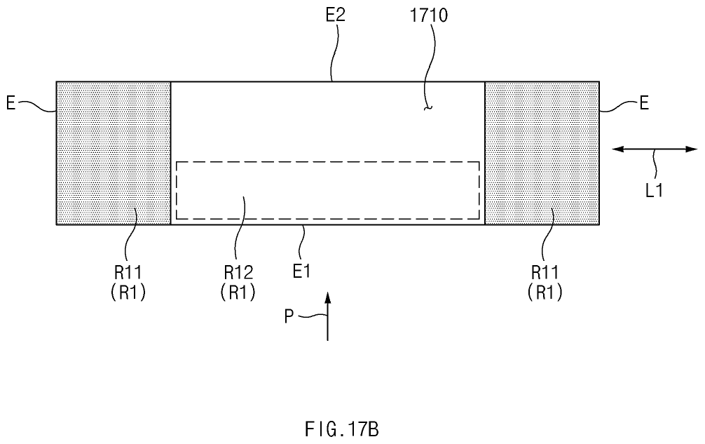

Referring to FIGS. 16A and 16B, a coupling member 1490 according to an embodiment may include a blocking member 1630. The blocking member 1630 may be disposed at an end 902E of a strap 14902. For example, the blocking member 1630 may be disposed on a second surface 902B of the end 902E. The blocking member 1630 may be disposed in a coupling groove 1410 of the end link 14901. For example, the blocking member 1630 may be disposed on a first region R1 of the coupling groove 1410. The blocking member 1630 may be positioned on a second surface 1410B of the coupling groove 1410. The blocking member 1630 may be interposed between the second surface 902B of the strap 14902 and the second surface 1410B of the coupling groove 1410. The blocking member 1630 may include a region that contacts the coupling groove 1410. For example, the blocking member 1630 may include a first region R1 that contacts the second surface 1410B of the coupling groove 1410. The first region R1 may be referred to as a partial region of the coupling groove 1410 or a partial region of the blocking member 1630 that contacts the coupling groove 1410. The blocking member 1630 may include a second region (e.g., a region other than the first region R1) that does not contact the coupling groove 1410, but is not limited thereto. Optionally, the second region of the blocking member 1630 may be removed through a process such as cutting after the strap 14902 and the end link 14901 are coupled to each other. Alternatively or optionally, after the strap 14902 and the end link 14901 are coupled together, the entire blocking member 1630 may be removed. Various examples of the first region R1 will be described below with reference to FIGS. 17A and 17B. The adhesive member 1520 may not be disposed in a space between the strap 14902 and the coupling groove 1410 corresponding to the first region R1.

In one embodiment, the blocking member 1630 may prevent the applied adhesive member 1520 from overflowing to the outside when the strap 14902 is inserted into the coupling groove 1410. Referring to FIG. 16A, the blocking member 1630 may include, for example, an adhesive tape 1630-1 (or an adhesive film 1630-1). The adhesive tape 1630-1 may include an adhesive tape made of resin such as polyimide, but is not limited thereto. The adhesive tape 1630-1 may be formed to be substantially transparent to reduce the influence on the design of the coupling member 1490, but is not limited thereto. Alternatively or additionally, referring to FIG. 16B, the blocking member 1630 may include a spacer 1630-2. The spacer 1630-2 may include metal or plastic. The spacer 1630-2 may include a portion to be disposed in the coupling groove 1410 (e.g., the second surface 1410B). The spacer 1630-2 may include a first region R1 that contacts the coupling groove 1410 (e.g., the second surface 1410B). The portion of the spacer 1630-2 disposed in the coupling groove 1410 may be formed to have a specified thickness to prevent the adhesive member 1520 from overflowing to the outside when the strap 14902 is inserted. The spacer 1630-2 may be removed after the end link 14901 and the strap 14902 are coupled to each other. The adhesive member 1520 may not be disposed in a space between the strap 14902 and the coupling groove 1410 corresponding to the first region R1.

Referring to FIGS. 16A and 16B, the blocking member 1630 is described as being disposed between the second surface 902B of the strap 14902 and the second surface 1410B of the coupling groove 1410, but is limited to thereto. Alternatively or additionally, the blocking member 1630 may be disposed between the first surface 902A of the strap 14902 and the first surface 1410A the coupling groove 1410. In addition, the blocking member 1630 may be provided in the form of the adhesive tape 1630-1 between the first surface 902A of the strap 14902 and the first surface 1410A of the coupling groove 1410, and provided in the form of the spacer 1630-2 between the second surface 902B of the strap 14902 and the second surface 1410B of the coupling groove 1410.

An insulating structure of the electronic device according to an embodiment may include the blocking member 1630.

FIG. 17A is a view showing an example of the first region according to an embodiment of the disclosure. FIG. 17B is a view showing an example of the first region according to an embodiment of the disclosure. In FIGS. 17A and 17B, an insertion direction P may be referred to as a direction in which the end 902E of the strap 14902 of FIGS. 15A and 15B is inserted into the coupling groove 1410 of the end link 14901. In FIGS. 17A and 17B, the insertion direction P may be referred to as a direction in which the coupling groove 1410 in FIG. 14A is depressed (e.g., a direction toward the side surface 901C). The insertion direction P may be substantially perpendicular to the longitudinal direction L1, but is not limited thereto.

Referring to FIGS. 17A and 17B, in one embodiment, the first region R1 may be formed on a surface 1710. The surface 1710 may be referred to as the first surface 1410A and/or second surface 1410B of the coupling groove 1410 in FIG. 16A. The surface 1710 may include two edges E opposite each other in the longitudinal direction L1, and a first edge E1 and a second edge E2 extending from the two edges E, respectively. The first edge E1 and the second edge E2 may oppose each other. The first region R1 may include first sub-regions R11 and a second sub-region R12.

Referring to FIG. 17A, the first sub-regions R11 of the first region R1 may be formed at both edges E of the surface 1710, respectively. The first sub-regions R11 may be partially formed at both edges E of the surface 1710. For example, the first sub-regions R11 may extend from the first edge E1 of the surface 1710. The first sub-regions R11 may extend toward the second edge E2. The first sub-regions R11 may extend to an arbitrary point between the first edge E1 and the second edge E2. In one embodiment, the first region R1 may further include a second sub-region R12. The second sub-region R12 may extend along the first edge E1 between the first sub-regions R11. The width (e.g., the length based on the insertion direction P) of the second sub-region R12 may be substantially the same as or smaller than that of the first sub-region R11.

Alternatively, the first sub-regions R11 may be formed on both edges E of the surface 1710. For example, referring to FIG. 17B, the first sub-regions R11 may extend from the first edge E1 of the surface 1710 to the second edge E2. In this case, the width of the second sub-region R12 may be smaller than that of the first sub-regions R11.

Referring to FIGS. 17A and 17B, the first sub-regions R11 and the second sub-region R12 are shown as being separated from each other, but the relationship is not limited thereto. For example, the first sub-regions R11 and the second sub-region R12 may be connected to each other.

FIG. 18A is a view showing an end link of a coupling member according to an embodiment of the disclosure. FIG. 18B is a cross-sectional view taken along line A-A′ in FIG. 18A according to an embodiment of the disclosure. FIG. 18C is a view showing the coupling member in which a strap is coupled to the end link according to an embodiment of the disclosure.

Referring to FIGS. 18A, 18B, and 18C, a coupling member 1890 according to an embodiment may include an end link 18901 in which a coupling groove 1810 is formed. The description of the coupling member 1490, the end link 14901, and the coupling groove 1410 may be applied to the coupling member 1890, the end link 18901, and the coupling groove 1810 in substantially the same, similar, or corresponding manner.

In one embodiment, the coupling groove 1810 may be formed in the end link 18901. The end 902E of the strap 14902 may be accommodated in the coupling groove 1810. In one embodiment, unlike the coupling groove 1410, both ends of the coupling groove 1810 along a longitudinal direction L1 may be closed. For example, an inner surface 1810A of the coupling groove 1810 may be formed to surround the entire circumference of the end 902E of the strap 14902. For example, the inner surface 1810A of the coupling groove 1810 may surround a first surface 902A and a second surface 902B of the end 902E, and a side surface 902C extending from an edge of the first surface 902A to an edge of the second surface 902B.

In one embodiment, a cross-sectional shape of the inner surface 1810A of the coupling groove 1410 may include, for example, a quadrangle, but is not limited thereto.

In one embodiment, the strap 14902 may be coupled to the coupling groove 1810 of the end link 18901. Since the coupling groove 1810 entirely surrounds the end 902E of the strap 14902, the adhesive member 1520 may not overflow to the outside even if the strap 14902 is inserted into the coupling groove 1810. Additionally or alternatively, a blocking member (e.g., the blocking member 1630 in FIGS. 16A and 16B) for preventing overflow of the adhesive member 1520 into a space between the inner surface 1810A of the coupling groove 1810 and the strap 14902 may be disposed.

An insulating structure of the electronic device according to an embodiment may include an adhesive member 1520 for joining the end link 18901 and the strap 14902.

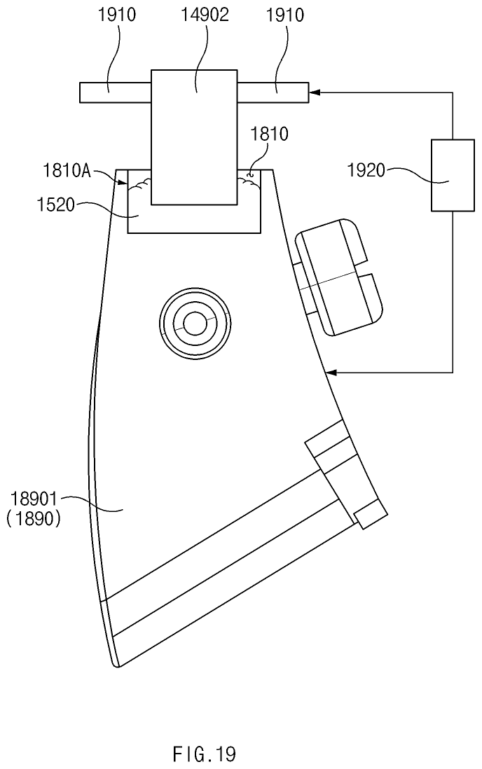

FIG. 19 is a view showing a method of inspecting insulation of a coupling member according to an embodiment of the disclosure.

Referring to FIG. 19, a jig 1910 may be electrically connected by contacting the strap 14902. An inspection device 1920 may be electrically connected to the jig 1910 and the end link 18901 (e.g., the end link 14901 in FIG. 14A). The inspection device 1920 may be electrically connected to the strap 14902 through the jig 1910. The inspection device 1920 may check whether current flows between the strap 14902 and the end link 18901. When the strap 14902 and the end link 18901 come into contact with the inner surface 1810A of the coupling groove 1410 due to poor application of the adhesive member 1520 or the like, current may flow between the strap 14902 and the end link 18901. Such an electrical path may reduce the accuracy of biometric information. Insulation inspection using the inspection device 1920 may be performed before the heat curing process for the adhesive member 1520.

A metal strap (e.g., the coupling member 1490 in FIG. 14A) of a wearable electronic device (e.g., the electronic device 100 in FIG. 1) according to an embodiment may include an end link (e.g., the end link 14901 in FIG. 14A) and a strap (e.g., the strap 14902 in FIG. 15B). A coupling surface (e.g., the coupling groove 1410 in FIG. 14A) may be formed on the end link. An end (e.g., the end 902E in FIG. 15B) of the strap may be coupled to the coupling surface. The insulating structure may include an adhesive member (e.g., the adhesive member 1520 in FIG. 16A) disposed between the coupling surface and the end of the strap. The end of the strap may be electrically separated from the coupling surface through the adhesive member.

In one embodiment, the end link may include a fixing member (e.g., the first fixing member 1450 in FIG. 14A) extending in a longitudinal direction (e.g., direction L1 in FIG. 14A). Both ends of the coupling surfaces that face each other based on the longitudinal direction may be open.

In one embodiment, the coupling surface may surround the entire end of the strap.

In one embodiment, the insulating structure may include a blocking member (e.g., the blocking member 1630 in FIG. 16A) disposed between the end of the strap and the coupling surface.

A wearable electronic device according to an embodiment may include a metal frame (e.g., the housing 110 in FIG. 1), a plurality of electrodes (e.g., the electrodes 11, 12, 13, and 14 in FIG. 4A), and a coupling member (e.g., the coupling member 1490 in FIG. 14A). The plurality of electrodes may be installed on the metal frame and configured to acquire biometric information by contacting a user's body. The coupling member may be coupled to the metal frame. The coupling member may include an end link (e.g., the end link 14901 in FIG. 14A), a metal strap (e.g., the strap 14902 in FIG. 15B), and an adhesive member (e.g., the adhesive member 1520 in FIG. 16A). The metal strap may be inserted into a coupling groove formed in the end link. The adhesive member may be disposed in the coupling groove to join the metal strap and the end link. The metal strap may be electrically separated from the end link through the adhesive member.

In one embodiment, the adhesive member may include an epoxy-based resin.

In one embodiment, the metal strap may include a mesh strap.

In one embodiment, the end link may include a side surface facing the metal frame (e.g., the side surface 901C in FIG. 14A). The coupling groove may be depressed in a direction toward the side surface.

In one embodiment, the end link may include a first fixing member (e.g., the first fixing member 1450 in FIG. 14A). The first fixing member may extend in a longitudinal direction (e.g., the direction L1 in FIG. 14A) and be detachably coupled to the metal frame. The coupling groove may be defined by a first surface (e.g., the first surface 1410A in FIG. 14A) and a second surface (e.g., the second surface 1410B in FIG. 14A). The first surface and the second surface may extend toward the side surface. The first surface and the second surface may be configured so that a distance between them becomes narrower as they approach the side surface. The coupling groove may be open in both directions opposed based on the longitudinal direction.

In one embodiment, the adhesive member may be at least partially disposed between an end of the metal strap inserted into the coupling groove and the first surface, and/or may be at least partially disposed between the end of the metal strap inserted into the coupling groove and the second surface.

In one embodiment, the end of the metal strap may be spaced apart from the first surface and the second surface.

The wearable electronic device according to an embodiment may further include a blocking member. The blocking member may be disposed between the end of the metal strap and the coupling groove.

In one embodiment, the blocking member may include an adhesive tape.

In one embodiment, the blocking member may include polyimide.

In one embodiment, the blocking member may include a first region (e.g., the first region R1 in FIG. 16A). The first region may include a region that is in contact with the coupling groove. The first region may be formed at both edges of the first surface and/or the second surface, which face each other based on the longitudinal direction.

In one embodiment, the blocking member may include a second region that is not in contact with the coupling groove.

In one embodiment, the first region may be partially formed at both edges.

In one embodiment, the first region may include first sub-regions (e.g., the first sub-regions R11 in FIGS. 17A and 17B) and a second sub-region (e.g., the second sub-region R12 in FIGS. 17A and 17B). The first region formed at both edges may be first sub-regions. The second sub-region may extend between the first sub-regions formed at both edges.

In one embodiment, the width of the second sub-region may be smaller than the width of the first sub-region.

In one embodiment, an end of the metal strap may be inserted into the coupling groove. An inner surface of the coupling groove may completely surround the end of the metal strap.

The wearable electronic device according to an embodiment may include a blocking member (e.g., the blocking member 1630 in FIG. 16A) disposed between the coupling groove and the metal strap.

In one embodiment, the first fixing member may be accommodated in a through hole (e.g., the through hole 1470 in FIG. 14C) formed in the end link. The first fixing member may include a first pin (e.g., the first pin 1452 in FIG. 14C), a second pin (e.g., the second pin 1453 in FIG. 14C), and an elastic member (e.g., the elastic member 1454 in FIG. 14C), a body (e.g., the body 51 in FIG. 5A), and an insulating member (e.g., the insulating member 65 in FIG. 6). The first pin and the second pin may be coupled to the metal housing. The elastic member may be disposed between the first pin and the second pin. The body may surround the first pin, the second pin, and the elastic member. The insulating member may be positioned between the through hole and the body and may surround the body.

An inspection method for a wearable electronic device (e.g., the electronic device 100 in FIG. 1) according to an embodiment may include a method of inspecting current flow of the metal strap and the end link using an inspection device (e.g., the inspection device 1920 in FIG. 19). The inspection device may be electrically connected to a jig (e.g., the jig 1910 in FIG. 19) in contact with the metal strap and the end link.

The electronic device according to various embodiments disclosed herein may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic device is not limited to those described above.

It should be appreciated that various embodiments of the disclosure and the terms used therein are not intended to limit the technological features set forth herein to particular embodiments and include various changes, equivalents, or replacements in corresponding embodiments. As used herein, each of phrases such as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. Terms such as “1st” and “2nd,” or “first” and “second” may be used to simply distinguish a corresponding component from another, and do not limit the components in other aspects (e.g., importance or order). It is to be understood that if an element (e.g., a first element) is referred to, with or without the term “operatively” or “communicatively,” as “coupled to,” “coupled to,” “connected with,” or “connected to” another element (e.g., a second element), it means that the element may be coupled to the other element directly (e.g., by wire), wirelessly, or via a third element.

As used in various embodiments disclosed herein, the term “module” may include a unit implemented in hardware, software, or firmware, and may interchangeably be used with other terms, for example, logic, logic block, part, or circuitry. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 1340) including one or more instructions that are stored in a storage medium (e.g., the internal memory 1336 or external memory 1338) that is readable by a machine (e.g., the electronic device 1301). For example, a processor (e.g., the processor 1320) of the machine (e.g., the electronic device 1301) may invoke at least one of the one or more instructions stored in the storage medium, and execute it. This allows the machine to be operated to perform at least one function according to the at least one instruction invoked. The one or more instructions may include a code generated by a compiler or a code executable by an interpreter. The machine-readable storage medium may be provided in the form of a non-transitory storage medium. Here, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments disclosed herein may be included and provided in a computer program product. The computer program product may be traded as a product between a seller and a buyer. The computer program product may be distributed in the form of a machine-readable storage medium (e.g., a compact disc read only memory (CD-ROM)), or be distributed (e.g., downloaded or uploaded) online via an application store (e.g., PlayStore™), or between two user devices (e.g., smartphones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the plurality of entities may be disposed separately from other components. According to various embodiments, one or more of the above-described components may be omitted, or one or more other components may be added. Alternatively or additionally, a plurality of components (e.g., modules or programs) may be integrated into a single component. In such a case, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A wearable electronic device comprising:
a metal frame;
a plurality of electrodes positioned on the metal frame to be in contact with a user's body and configured to form an electrical signal path with the user's body in contact to acquire biometric information;
a metal strap connected to the metal frame; and
an insulating structure,
wherein the insulating structure is configured to electrically separate the metal strap and the metal frame to reduce leaking of the electrical signal to the metal frame through the metal strap.

2. The wearable electronic device of claim 1, wherein the insulating structure includes:
a body accommodated in a through hole formed in the metal strap;
a first pin and a second pin disposed at both ends of the body and connected to the metal frame; and
an insulating member surrounding the body so that the metal strap and the metal frame are electrically separated.

3. The wearable electronic device of claim 1, wherein the insulating structure includes:
a body accommodated in a groove formed in the metal strap;
a first pin and a second pin disposed at both ends of the body and connected to the metal frame; and
an insulating member surrounding the body so that the metal strap and the metal frame are electrically separated.

4. The wearable electronic device of claim 3,
wherein the groove extends from a first side surface of the metal strap to a second side surface opposite the first side surface in a longitudinal direction,
wherein a cross-sectional shape of the groove cut in a direction perpendicular to the longitudinal direction has an open circular shape,
wherein the metal strap includes a first surface extending from a partial edge of the first side surface to a partial edge of the second side surface,
wherein the first side surface faces the user's body while the user is wearing the wearable electronic device, and
wherein the groove is formed in the first surface.

5. The wearable electronic device of claim 1,
wherein the metal strap includes an end link connected to the metal frame, and
wherein the end link includes an insulating material so that the metal strap and the metal frame are electrically separated.

6. The wearable electronic device of claim 1,
wherein the metal strap includes an end link connected to the metal frame, a center link connected to the end link, and a side link connected to the center link, and
wherein at least some of the end link, the center link, or the side link are made of an insulating material.

7. The wearable electronic device of claim 1, further comprising:
an insulating layer,
wherein the metal frame includes a front surface, a rear surface opposite the front surface, and a side surface extending from the front surface to the rear surface, and
wherein the insulating layer is formed on at least a portion of the side surface of the wearable electronic device.

8. The wearable electronic device of claim 7,
wherein the metal frame includes a first protrusion and a second protrusion extending from the side surface,
wherein the metal strap is connected between the first protrusion and the second protrusion, and
wherein the insulating layer is formed in a region between the first protrusion and the second protrusion on the side surface.

9. The wearable electronic device of claim 1, further comprising:
a printed circuit board; and
at least one element disposed on the printed circuit board and electrically connected to a ground portion of the printed circuit board,
wherein the metal frame is electrically connected to the at least one element, and
wherein the at least one element includes a capacitor.

10. The wearable electronic device of claim 1,
wherein the plurality of electrodes include a first portion made of an electrically conductive material and a second portion made of an electrically insulating material,
wherein the first portion is spaced apart from the metal frame and is configured to form an electrical signal path with a user's body in contact, and
wherein the second portion is connected to the first portion, at least partially accommodated in the metal frame, and is configured to electrically insulate the first portion and the metal frame.

11. The wearable electronic device of claim 1,
wherein the metal strap includes an end link and a strap,
wherein a coupling surface is formed on the end link,
wherein an end of the strap is coupled to the coupling surface,
wherein the insulating structure includes an adhesive member disposed between the coupling surface and the end of the strap, and
wherein the end of the strap is electrically separated from the coupling surface through the adhesive member.

12. The wearable electronic device of claim 11,
wherein the end link includes a fixing member extending in a longitudinal direction, and
wherein both ends of the coupling surfaces that face each other based on the longitudinal direction are open.

13. The wearable electronic device of claim 11, wherein the coupling surface surrounds all of the end of the strap.

14. The wearable electronic device of claim 11, wherein the insulating structure includes a blocking member disposed between the end of the strap and the coupling surface.

15. A method of acquiring the biometric information by the wearable electronic device of claim 1, the method comprising:
determining whether the metal strap is fastened to a user;
detecting the biometric information based on the determining;
correcting the detected biometric information based on a pre-stored reference value of biometric information; and
displaying the corrected biometric information.
